(19) United States
(12) Patent Application Publication
(10) Pub. No.: US 2024/0181204 A1
(43) Pub. Date: Jun. 6, 2024

(54) IMPLANTABLE MEDICAL DEVICES AND TUBING

(71) Applicant: FREEFLOW MEDICAL DEVICES LLC, Lancaster, PA (US)

(72) Inventors: Saibal BANDYOPADHYAY, Lancaster, PA (US); Andrew K. JONES, Lancaster, PA (US)

(21) Appl. No.: 18/284,946

(22) PCT Filed: Mar. 31, 2022
(86) PCT No.: PCT/US2022/022951

§ 371 Date: Sep. 29, 2023

Related U.S. Application Data
(60) Provisional application No. 63/169,047, filed on Mar. 31, 2021.
(60) Provisional application No. 63/310,066, filed on Feb. 14, 2022.

(51) Int. Cl. A61M 25/00; A61L 29/08; A61L 29/14; A61M 1/36; A61M 27/00

(52) U.S. Cl. CPC A61M 25/0026; A61L 29/085; A61L 29/146; A61M 1/3661; A61M 27/006; A61M 25/0009; A61M 2025/0037; A61M 2025/0046

(57) ABSTRACT

The present disclosure provides tubing comprising porous fluoropolymers for use in non-medical and medical applications along with methods of making the tubing. Also described are medical devices including catheters and shunts comprising the tubing. Specifically, a catheter, cannula, drain, shunt, or totally implantable venous access port comprising a section of tubing with at least two lumens separated by septa, as well as an outer wall and a fluorinated liquid. Wherein the outer wall and the septa are a porous fluoropolymer and the fluorinated liquid is absorbed into the pores of both the septa and the outer wall of the tubing.

### 1.0 BACKGROUND

Catheters used for long term medical treatments that are inserted into a vein or artery of a patient/subject, such as hemodialysis catheters (HDC), have a high complication rate because of infections and dysfunction. Within hours of catheter placement, a fibrin sheath starts covering the catheter from the point of insertion and may eventually cover most, if not all, of the entire catheter surface. Fibrin sheath formation and intraluminal thrombosis are primary causes of catheter dysfunction, which in the case of a hemodialysis catheter is defined as the inability to sustain a blood flow rate of at least 300 ml/min through the catheter. The incidence of catheter dysfunction ranges from 0.5-3.42 episodes per 1.000 catheter-days.

Catheter dysfunction due to intraluminal thrombosis and fibrin sheath formation impairs adequate dialysis and often requires salvage by instillation of a thrombolytic agent into the catheter lumen and/or catheter exchange. In situ treatments to restore catheters to adequate functional flow rates. thereby avoiding catheter exchange, include the use of fibrinolytics locally or systemically. Heparin coated catheters, such as HDC, fail to resist fibrin sheath formation upon long term implantation.

Fibrin sheath formation has a close relationship with pathogen colonization and biofilm formation on the catheter surface. The entry of pathogens into the bloodstream through extraluminal and intraluminal routes and the seeding of pathogens that develop biofilm on the catheter surface cause catheter-related bloodstream infection (CRBSI). In the US, annual hemodialysis (HD) treatment costs ˜$89.000 per patient, with a total cost of ˜$42 billion. In 2016, 80% of patients used a catheter at the initiation of HD, and 18.6% of all HD patients in the US were using a catheter in 2017. Researchers have explored coating commercialized HDCs with antibiotic-impregnated coatings to reduce CRBSI. The most successful coatings to date have been minocycline-rifampin/rifampicin (GlideSpectrum™. Cook Medical) and chlorhexidine coatings (ARROWgard Blue™; Arrow International, Reading, PA. USA). These active antimicrobial-releasing coatings marginally reduce CRBSI, but no catheter coating has demonstrated clinically significant differences in the overall rates of bloodstream infections or mortality.

Some clinical practice strategies have reduced infection rates associated with HDC. Among these are the use of antibiotic-coated HDC for high-risk patients, and antibiotic lock solutions, which are not recommended for long-term use. In addition, the CDC-recommended the use of aseptic techniques, with the use of topical antimicrobial agents being recommended only in hospital settings.

In view of the foregoing, new approaches are still needed to improve patient care and reduce the catheter complication rate due to infection and dysfunction.

### 2.0 SUMMARY

Although technologies including both heparin and antimicrobial coatings are currently available, no single solution addresses fibrin sheath formation, intraluminal thrombosis, and CRBSI. Moreover, there are no presently available solutions to prevent dysfunction of medical devices including catheters that can also successfully prevent both thrombosis and infection. The bio-passive surfaces and surface coatings of the tubing and medical devices (e.g., catheters) described herein offer an avenue to reduce the incidence of medical device (e.g., catheter) dysfunction, thrombosis, and infection without the risk of creating antibiotic-resistant strains.

The medical devices, including catheters (e.g., HDCs), of the present disclosure incorporate a slippery surface or coating (e.g., a slippery liquid-infused porous surface “SLIPS” or SLIPS-like coating, see, e.g., WO 2012/100100) and resist the attachment of fibrin (and accordingly fibrin sheath formation), thrombus formation, and pathogen attachment and colonization. The slippery coating is formed by a fluorinated liquid, e.g., a liquid fluorocarbon or perfluorocarbon, and may be water repellant (e.g., having a water roll off angle less than about 10° or less than about 5°), hydrophobic (having a water contact angle greater than 90° at 20° C., or even omniphobic (repelling water and non-fluorinated liquids including oils). The catheters, and particularly the portions with a slippery omniphobic surface or coating, are comprised of one or more fluoropolymers and/or perfluoropolymers. Where the fluoropolymers are to retain fluorinated liquids within the polymer they may be porous fluoropolymers (e.g., expanded or electrospun fluoropolymers). Expanded fluoropolymers are denoted by a lower case “e” preceding the polymer name or acronym (e.g., expanded PTFE is ePTFE). All expanded fluoropolymers as used herein are porous, with at least a portion of the pores having an open structure into which fluorinated liquids (e.g., perfluorinated liquids) may enter. Some fluoropolymers or perfluoropolymers that can be used include, but are not limited to, polytetrafluoroethylene (PTFE), fluorinated ethylene propylene (FEP), ethylene fluorinated ethylene propylene (EFEP), perfluoro alkoxy alkane (PFA), polyvinylidene fluoride (PVDF), ethylene tetrafluoroethylene (ETFE) and porous versions thereof. When a fluorinated liquid is applied on the fluoropolymers or perfluoropolymers it coats the surface producing the slippery coating. Utilizing porous fluoropolymers and/or porous perfluoropolymers permits the fluorinated liquid to be infused into (wick into) the fluoropolymers or perfluoropolymers, thereby providing not only a slippery omniphobic surface that is bio-passive, resisting, among other things, cell attachment, but also a reservoir of fluorinated liquid that maintains the surface properties.

Among the innovations provided by the design of the tubing, and the catheters, cannulas, shunts, and other medical devices employing the dual or multi lumen tubing described herein is the use of septa (wall) between some or all of the lumens that comprise a porous fluoropolymer. By using septa comprising a porous fluoropolymer the catheters of the present disclosure provide a reservoir for the fluorinated liquid that can migrate from the septa to the external wall that may keep the surface of the catheter coated in a “slippery” state for a longer period of time than without a porous septa reservoir. Using a porous septa permits the incorporation of a reservoir without having to increase the catheter's wall thickness to provide a reservoir for the fluorinated liquid. The use of porous septa as a reservoir also permits the preparation of catheters without substantially altering the flexibility of the tubing relative to otherwise equivalent tubing lacking the porous septa. Another feature that may be incorporated into tubing, and the catheters, cannulas, shunts, and other implantable medical devices described herein incorporating the tubing, is the addition of one or more (e.g., two or more) reservoir lumens to contain a supply of fluorinated liquid. Reservoir lumens of implantable medical devices are optionally accessible through, for example, the external portion of the implanted medical device (e.g., catheter, cannula, drain, etc.), and may be refilled through the external portion. The lumen employed as a reservoir may be formed by closing off (e.g., sealing partially or completely) one or more (e.g., two or more) lumens of the catheter other than those lumens used to access a fluid or tissue of a subject or patient (e.g., to access a subject's blood, cerebrospinal fluid, etc.). Where the reservoir is to be refillable and a portion of the medical device is not implanted, such as in the case of a catheter or cannula, it may be fitted with a septum or valve through which fluorinated liquid can be introduced into the portion of the device external to the patient/subject.

The catheters, cannulas, drains, and other medical devices described herein can provide resistance to fibrin sheath and/or thrombosis formation for an extended period of time (e.g., less than six percent (6%) of the surface area being covered by fibrin sheathing and/or thrombi at ninety (90) days post implantation). While the catheter resists fibrin sheath formation and the formation of thrombi, the wall of the vessel into which the catheter is inserted seals to the catheter providing a stable implant. The surfaces of the medical devices formed from the tubing provided herein also resist colonization by microorganisms and thereby impact the occurrence of CRBSI.

### 3.0 BRIEF DESCRIPTION OF THE DRAWINGS

FIG. 1 shows a portion of a dual lumen catheter 1 having a portion with optionally solid walls 2 and a portion with porous walls 3 at A. The expanded section in B shows the segment 4 where the portion with a wall that is optionally nonporous (e.g., has a nonporous liner) transitions to the portion with a porous exterior layer or porous outer wall (exterior wall) 5 into which a slippery liquid has been infused. Fittings that form a fluid tight seal (e.g., Luer locks) are indicated at 7, and optional clamps at 8. Although the wall (septa) between the lumens 6 is shown as a straight structure, the catheter may have a curved wall between the two lumens (e.g., giving rise to a crescent shaped lumen as in FIG. 4 at e and f). All or part of the septa between the lumens may comprise porous material that can act as a reservoir, which is in fluid communication with the exterior porous layer. Furthermore, the structure of the lumens within the catheter may be one of concentric tubes, with one tube nested completely inside the lumen of the second tube, which has a larger lumen.

FIG. 2 shows an exemplary process of preparing a dual lumen catheter of the present disclosure.

FIG. 3 shows part of an exemplary process for preparing a lined dual lumen section of tubing (e.g., for use in preparing a catheter or other device of the present disclosure). In the process liners 31 are slid into the lumens 35 of a dual lumen tube 30 (e.g., extruded as one piece) comprised of a porous fluoropolymer. The process begins at (a.) with the liners 31 slid over a pair of D-shaped mandrels 33 that are arranged so that they are spaced apart and there is a gap 34 between the mandrels to accommodate the septum between the lumens 35. At (b.) mandrels 33 and liners 31 are fully inserted into the lumens of tube 30. At (c.) the mandrels 33 are withdrawn leaving the liners 31 in the lumens of tube 30. At (d.) two views of the dual lumen section showing part of the porous fluoropolymer tube 30 cutaway and liners 31 exposed. Part (d.) also shows a cross sectional view of the tubing indicating the location of the porous fluoropolymer tube 30, liners 31, and lumens 35. The same technique may be used with triple-lumen and quadruple-lumen tubing used for catheters, cannulas, etc. Where two or more lumens are present, not all of the lumens are required to be lined. In some cases one lumen is left unlined. Reservoir lumens, when present, are generally left unlined permitting the fluorinated liquid in the reservoir direct access to the porous fluoropolymer.

FIG. 4 shows fourteen views of a dual lumen tube that may be employed in non-medical and medical applications e.g., as part of a catheter, cannula, shunt, or line inserted or implanted in a patient or subject. The cross-sectional views indicate the location of the outer surface of the catheter 20, lumen(s) 21, and the porous outer wall (exterior wall) 22. The porous septa dividing the lumens 23 and 25 may act as a reservoir for one or more (at least one) fluorinated liquids. The tubing may have a nonporous liner 24 that is separated by a nonporous septa 27. Fluorinated liquid may also be in reservoirs such as lumen 26 in structure (g), or luminal spaces 29 formed between outer wall 22 and an infolding of the liner 28. Fluorinated liquid can migrate to the outer wall 22 from porous septa 23 or 25 as shown in structures (a)-(g), (i) and (j) as indicated by the small solid arrows extending from the septa to the outer walls. Where a lumen is formed by an infold of the liner 28 and the outer wall 22, fluorinated liquid introduced into the lumen may also flow into the outer wall as indicated in structures (i) and (j). Arrows representing the flow of fluorinated fluid are omitted from (e) through (h) and (k) through (n) for clarity. The solid line indicating the outer surface of the catheter at 20 is not intended to indicate the surface is covered by a nonporous material.

FIG. 5 provides a diagram of test and control catheter placement as described in Example 1.

FIG. 6 show an image of the control (A) and test (B) catheters after explant following 90 days of implantation. A large sheath of tissue (fibrinous sheath) covers a substantial portion of the control catheter compared to the test catheter. The last panel of FIG. 6, panel (C), shows a close-up of the transition between the proximal portion and distal portion of the test catheter from Example 1 following 90 days of implantation.

FIG. 7 provides images of the distalmost end of the catheters implanted in Example 1. At A the hole at the distalmost end of the control catheter is completely obstructed following 90 days of implantation. The hole at the distalmost end of the test catheter shown at B is unobstructed after 90 days of implantation.

### 4.0 DETAILED DESCRIPTION

### 4.1 Definitions

The terms “patient” and “subject” are used interchangeably throughout this disclosure.

Unless indicated otherwise. “fluoropolymer” includes perfluoropolymer.

Unless indicated otherwise, the term “consisting essentially of” as applied to chemical compositions means more than 50% of the composition by weight.

Unless indicated otherwise, the term “substantially” is intended to encompass both “wholly” and “largely but not wholly.”

### 4.2 Design and Composition

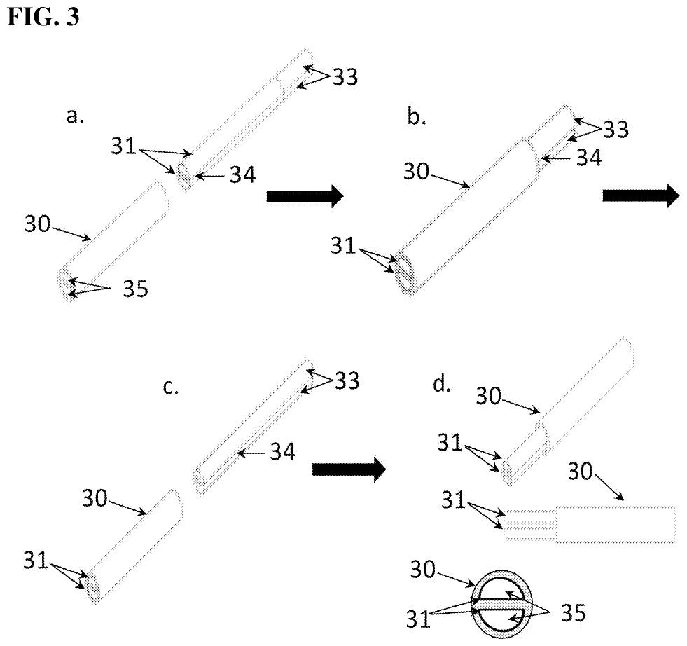

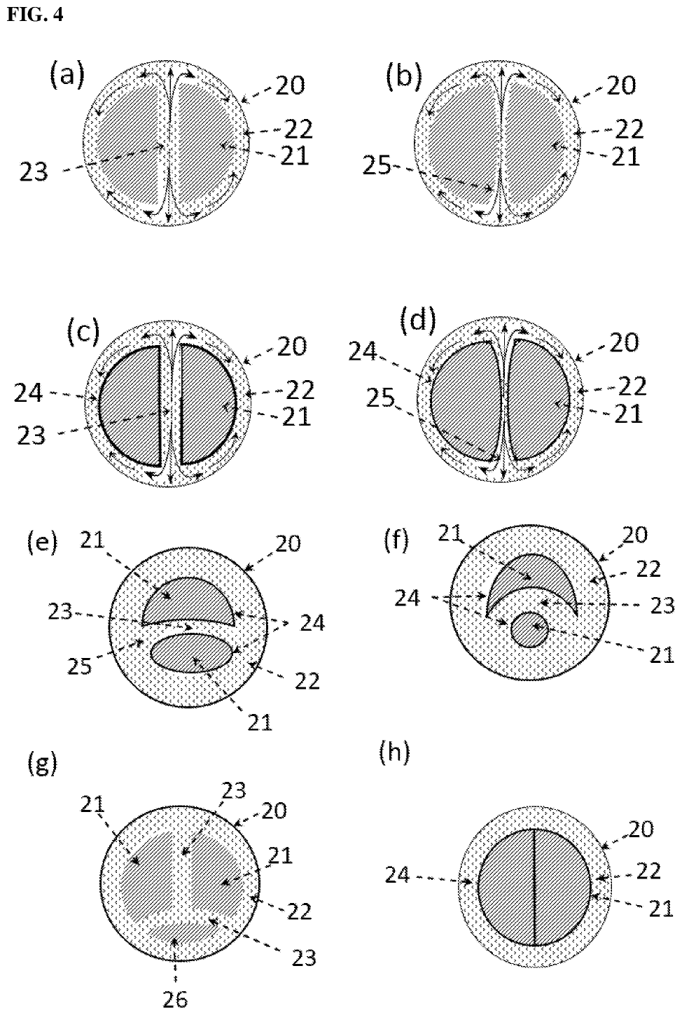

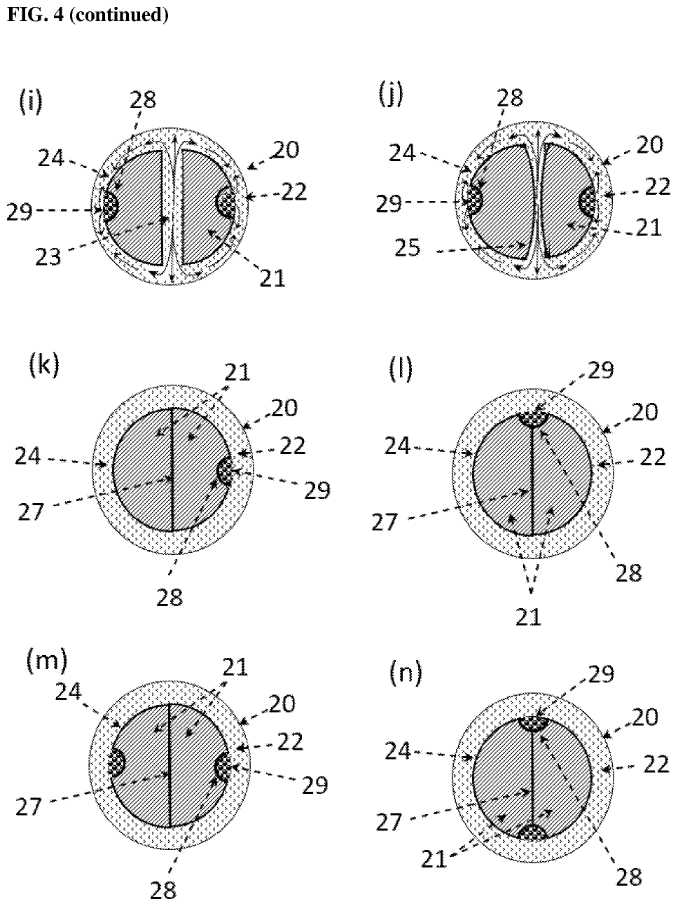

The tubing and medical devices such as catheters, cannulas, and drains described herein having, for example, a dual lumen structure allow fluid (e.g., blood) inflow and outflow from a patient's body using, for example, a catheter and a single site of insertion. The same concepts applied to the dual lumen tubing and medical devices (e.g., catheters) can be applied to multiple lumen (e.g., triple-lumen, or quadruple-lumen tubing) and medical devices incorporating such tubing. For example, in double- or triple-lumen hemodialysis catheters the arterial lumen serves for blood delivery to the patient/subject, and the other lumen(es) (the venous lumen(es)) serve to carry flow from the patient/subject to dialysis equipment. To give flexibility to tubing and medical devices (e.g., catheters, cannulas, shunts, drains, etc.), the bulk material used to form at least a portion of the device designed to be inserted into the patient or subject (the distal portion of a catheter as used herein) may be comprised of a porous (e.g., expanded or electrospun) fluoropolymer or perfluoropolymer, such as porous PVDF or porous PTFE, or are coated with a layer of porous fluoropolymer. In those instances where a medical device comprises tubing with one or more lumens, the lumens may be lined with a nonporous polymer, fluoropolymer or perfluoropolymer liner (lining). For example, either one or both of the inner lumens of a dual lumen catheter may be lined. Where both of the lumens comprise a liner (lining), the catheter may be formed from a nonporous dual lumen tube comprised of a polymer (e.g., polyurethane), fluoropolymer, perfluoropolymer, or a combination thereof, around which the porous fluoropolymer may be formed or applied (see, e.g., FIG. 4, structure h). Alternatively, tubing with an inner double lumen may be constructed by combining two nonporous fluoropolymer or perfluoropolymer tubes each having a single lumen as illustrated in FIG. 2. When combined, the two nonporous tubes form a double lumen structure that may comprise an adherent material and/or an optional layer of intervening material (e.g., a porous material layer that acts as a spacer) 13 along the point of contact (see, e.g., FIG. 2). The same type of process may be utilized to prepare multiple (e.g., triple) lumen catheter structures. Liners (e.g., of nonporous polymer, fluoropolymer or perfluoropolymer) may also be slid into dual lumen tubing as in FIG. 3. Regardless how the multiple (e.g., double, triple, quadruple, multiple) lumen structure is formed, once it is prepared it has or is coated with a porous fluoropolymer exterior layer.

The tip of a medical device comprising a section of tubing (e.g., dual or triple lumen tube of a catheter, cannula, drain, shunt, etc.) can be engineered further by creating holes in the tube's side wall that penetrate into a lumen, thereby placing the lumen in fluid communication with the exterior of the catheter through the holes. Holes made in a side wall may also pass through a septum between any two lumens additionally placing them in fluid communication. The holes in the lumens may be made in the same region (distance range as measured from the end of the tip) of the device (e.g., tip of a catheter cannula, drain, shunt, etc.). Alternatively, holes through the side wall providing fluid communication with different lumens may be made at different distances from the tip. For example, holes in the arterial lumen (first lumen of a dual lumen catheter) and the venous lumen (second lumen of a dual lumen catheter) may be made in different regions (in a different range of distances) from the end of the catheter's tip.

At least a portion (e.g., all) of the tubing or medical device comprising the tubing described herein inserted into a patient comprises, consists essentially of, or consists of a porous fluoropolymer/perfluoropolymer outer wall or outer (exterior) layer. Septa between lumens (walls between the lumens) may also comprise, consist essentially of, or consist of a porous fluoropolymer/perfluoropolymer (e.g., the same porous fluoropolymer used for the outer layer). For example, at least a portion of the catheter described herein (e.g., the distal portion of the HDCs designed to be inserted into a patient) comprises, consists essentially of, or consists of a porous fluoropolymer/perfluoropolymer outer wall or outer layer. A variety of porous fluorinated and/or perfluorinated polymers may be used to form that outer layer. The porous fluorinated and/or porous perfluorinated polymer may be expanded polytetrafluoroethylene (ePTFE) or electrospun PTFE. The porous fluorinated and/or perfluorinated polymer (fluoropolymer and/or perfluoropolymer) may be expanded PVDF (ePVDF). The porosity of the fluoropolymer or perfluoropolymer may be infused with a fluorinated liquid by, for example, contacting the liquid with the porous fluoropolymer or perfluoropolymer.

The outer surface of tubing of the present disclosure, including sections of tubing of the present disclosure incorporated into medical devices, may have a defined roughness, which is assessed in the absence of the fluorinated liquid unless stated otherwise. Roughness may be measured by Coherence Scanning Interferometry using a NewView™ 9000 instrument by Zygo Corporation, (Middlefield, CT) with a 50× objective at 1× zoom. The Software develop ratio (Sdr), which is the percentage of a definition region's additional surface area contributed by the texture as compared to the planar area of the definition region, may be employed as a measure of roughness. Measured Sdr values can range from 0.00001 to greater than 10.0. Some Sdr ranges for the outer surface of tubing of the present disclosure include from about 0.1 to about 4.0. For example, the outer surface of the tubing may have an Sdr from about 0.1 to about 0.25 or from about 0.25 to about 0.5. The outer surface of the tubing may, for example, have an Sdr from about 0.5 to about 1.0 or from about 1.0 to about 2.0. The outer surface of the tubing may also have an Sdr from about 2.0 to about 3.0 or from about 3.0 to about 4.0. For example, some ePTFE samples may have Sdr values from about 0.25 to about 0.35 or from about 0.55 to about 0.75, and some electrospun PTFE samples may have Sdr values in the range of about 2.5 to about 3.5.

Porous fluoropolymer or perfluoropolymer (e.g., ePVDF or ePTFE) used to prepare the tubing and medical devices of the present disclosure may have a density in a range selected from: 0.3-0.4 grams per cubic centimeter (g/cc), 0.4-0.5 g/cc, 0.5-0.6 g/cc, 0.6-0.7 g/cc, 0.7-0.8 g/cc, 0.8-0.9 g/cc, 0.9-1.0 g/cc, 1.0-1.1 g/cc, 1.1-1.2 g/cc, 1.2-1.3 g/cc, 1.3-1.4 g/cc, 1.4-1.8 g/cc and 1.8 to 1.9 g/cc. As discussed above, the use of porous fluoropolymers permits fluorinated liquids to be absorbed into and occupy the void space of the pores. The amount of void space in ePTFE or electrospun PTFE per gram of material is described in Table 1 along with the approximate maximum weight of an exemplary fluorinated liquid, perfluorodecalin, having an approximate density of 1.92 g/cc at 25° C.

TABLE 1

| | | | Maximum grams of |
|---|---|---|---|
| | | Reservoir Volume in | perfluorodecalin |
| Density grams/cubic | Approximate | cc, per gram | per gram of |
| centimeter (cc) | % Void Space | of tubing | tubing at 25° C. |
| 2.2 | 0 | 0 | 0 |
| 1.9 | 14 | 0.14 | 0.14 |
| 1.8 | 18 | 0.18 | 0.19 |
| 1.7 | 23 | 0.23 | 0.26 |
| 1.6 | 27 | 0.27 | 0.32 |
| 1.5 | 32 | 0.32 | 0.41 |
| 1.4 | 36 | 0.36 | 0.49 |
| 1.3 | 41 | 0.41 | 0.61 |
| 1.2 | 45 | 0.45 | 0.72 |
| 1.1 | 50 | 0.50 | 0.87 |
| 1.0 | 55 | 0.55 | 1.06 |
| 0.9 | 59 | 0.59 | 1.43 |

A porous fluoropolymer (e.g., ePTFE) used to prepare the tubing and/or medical devices of the present disclosure may have a density from about 0.9 to about 1.0 g/cc or about 1.0 to about 1.1 g/cc. A porous fluoropolymer used to prepare the tubing and/or medical devices of the present disclosure may have a density from about 1.0 to about 1.2 g/cc or about 1.2 to about 1.4 g/cc. A porous fluoropolymer used to prepare the tubing and/or medical devices of the present disclosure may have a density from about 1.4 to about 1.5 g/cc or about 1.5 to about 1.6 g/cc. A porous fluoropolymer used to prepare the tubing and/or medical devices of the present disclosure may have a density from about 1.4 to about 1.6 g/cc or about 1.6 to about 1.8 g/cc. A porous fluoropolymer used to prepare the tubing and/or medical devices of the present disclosure may have a density from about 1.7 to about 1.9 g/cc or about 0.9 to about 1.9 g/cc.

The porous fluoropolymer (e.g., ePTFE) may provide a maximum reservoir volume (open pore space that is accessible to, and can be filled with, fluorinated liquid) from about 0.1 to about 0.2 cc/g or about 0.2 to about 0.3 cc/g. The porous fluoropolymer may provide a maximum reservoir volume from about 0.2 to about 0.4 cc/g or about 0.3 to about 0.5 cc/g. The porous fluoropolymer may provide a maximum reservoir volume from about 0.4 to about 0.5 cc/g or about 0.5 to about 0.6 cc/g. The accessible pore volume may be less than the maximum volume of the pores present in the fluoropolymer (based on the difference in density between the solid and porous polymer) as some pores may not be accessible. The accessible volume may be determined by placing a weighed sample of the polymer under vacuum and then exposing it to perfluorodecalin (vacuum infiltrating it with perfluorodecalin) and reweighing it to determine the amount (weight) of perfluorodecalin taken into the polymer sample. The density of perfluorodecalin can be used to calculate the volume of the porous fluoropolymer accessible to fluorinated liquid per gram of porous fluoropolymer using vacuum infiltration with the perfluorodecalin.

The fluorinated liquid acts in combination with the porous fluoropolymer not only to form a biopassive surface, but also to lubricate the portion of the catheter having a porous fluoropolymer surface. In the present disclosure the fluorinated liquids (e.g., perfluorinated liquids) include, but are not limited to, one or more liquids selected from: perfluoropropane, perfluorobutane, perfluoropentane, perfluorohexane, perfluorooctane, perfluorodecalin, perfluoroperhydrophenanthrene, perfluorooctylbromide, perfluorotributylamine, perfluorotripentyl amine, poly(hexafluoropropylene oxide), 1H,4H-perfluorobutane, 1H-perfluoropentane, HFA 134a™, HFA227ea™, methyl perfluorobutylether, methyl perfluoropropyl ether (3M Novec 7000™), 2,2,2-trifluoroethanol, perfluoro-poly-propylene oxide (Krytox oil) and combinations thereof. The fluorinated liquids may be fluorocarbons, perfluorocarbons, or mixtures comprising, consisting essentially of, or consisting of fluorocarbons and/or perfluorocarbons.

Fluorinated and/or perfluorinated liquids suitable for use with the tubing, and/or medical devices (e.g., catheters, cannulas, shunts, drains, etc.) of the present disclosure include those with boiling point ranges above and below the physiological temperature of 37° C. Fluorinated liquids, or mixtures of fluorinated liquids (e.g., mixtures of fluorinated liquids and/or perfluorinated liquids), for use with the catheters of the present disclosure may be selected to have a boiling point greater than 30° C., greater than 35° C., greater than 40° C., greater than 45° C., or greater than 50° C. The boiling points of some fluorinated liquids, or mixtures of fluorinated liquids, may be in a range selected from less than 30° C. from 30° ° C. to 60° C., from 60° C. to 100° C., from 100° C. to 200° C., from 200° C. to 220° C. or greater than 220° C. The boiling points of some individual fluorinated liquids are provided in the table that follows.

TABLE 2
Liquid perfluorocarbons and their boiling points

| Perfluorinated liquid | Boiling point ° C. | Perfluorinated liquid | Boiling point ° C. |
|---|---|---|---|
| perfluoropropane | −36.7 | perfluorodecalin | 142 |
| perfluorobutane | −1.7 | perfluoroperhydrophenanthrene | 215 |
| perfluoropentane | 28.0 | perfluorooctylbromide | 142 |
| perfluorohexane | 56.0 | perfluorotributylamine | 178 |
| perfluoroheptane | 56.0 | perfluorotripentylamine | 215 |
| perfluorooctane | 103.0 | Krytox oil | |

Fluorinated liquids include and may be selected from the group consisting of: perfluoropropane, perfluorobutane, perfluoropentane, perfluorohexane, perfluorooctane, perfluorodecalin, perfluoroperhydrophenanthrene, perfluorooctylbromide, perfluorotributylamine, perfluorotripentylamine, poly(hexafluoropropylene oxide) and combinations thereof.

When present, the lining(s) of lumens present in tubing or medical devices described herein may be comprised of one or more solid (nonporous) polymers that can block the flow of fluids in and out of the lumens through walls that are lined. For medical devices, the polymers are preferably biocompatible with biological fluids including, but not limited to, mammalian blood, cerebrospinal fluid, and/or urine. Examples of such solid polymers include polyurethanes, silicones, and fluoropolymers and/or perfluoropolymers (e.g., fluoropolymer blends). The liners may, for example, be made of PTFE, FEP, PFA, PVDF, EFEP, or ETFE. The liners may be made of PTFE or FEP. As a lining material, FEP is softer and more flexible than PTFE. Using fluoropolymers and/or perfluoropolymers allows fluorinated liquids (e.g., fluorocarbon and/or perfluorocarbon) to coat the liner's surface forming, for example, a slippery surface that may be hydrophobic or omniphobic.

Liners present in the tubing described herein, and liners present in sections of the tubing described herein incorporated into medical devices (e.g., catheters, cannulas, shunts, drains, etc.), including the dual lumen catheters (e.g., dual-lumen HDCs) of the present disclosure with a DD structure, may have any one or more lumens lined with a nonporous liner. Each of the nonporous liners may comprise an independently selected polymer, fluoropolymer, and/or perfluoropolymer. For example, the nonporous polymer, nonporous fluoropolymer, or nonporous perfluoropolymer may be selected from the group consisting of polymers that comprise, consist essentially of, or consist of: PFA, FEP, PTFE or PVDF, EFEP, ETFE, fluoroelastomer, perfluoroelastomer, and fluorosilicone rubber. Layers of intervening material that may act as spacers between any of the liners may be comprised of any one or more of those materials, and in addition, may act as a thermoplastic adhesive (see, e.g., FIG. 2, item 13 where the intervening layer of material may be a porous spacer).

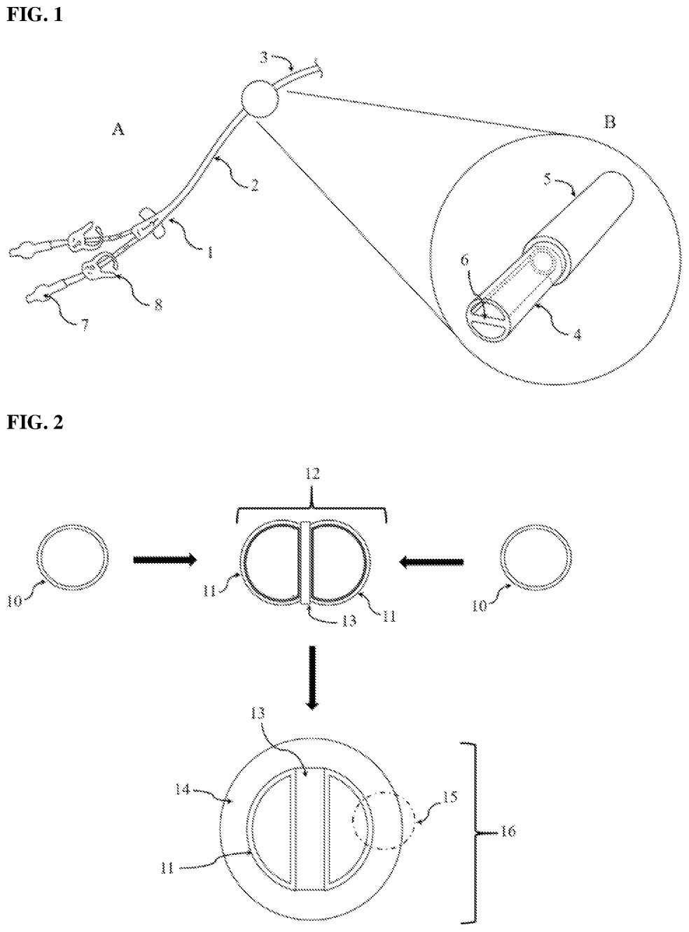

Some versions of the tubing and medical devices (e.g., catheters, cannulas, shunts, drains, etc.) described herein, including some HDCs, have a dual lumen structure, where the individual inner lumens are each a half-circle or “D”, with the flat surfaces of the half-circles or Ds facing each other (see FIG. 1). Such an inner lumen design is termed a “DD” structure. In tubing with a DD structure, or devices employing tubing with a DD structure (e.g., catheters), the inner liner of one or both of the “D” shaped lumens may be prepared out of (e.g., lined with): nonporous fluoropolymers or their blends (e.g., PTFE, FEP, PFA, PVDF, EFEP, ETFE or combinations thereof); or non-fluorinated materials (e.g., silicones or polyurethanes). The flat surfaces of the “D” shaped lumen liners may be joined by an adherent material such as a polymer or fluoropolymer (e.g., a thermoplastic polymer or fluoropolymer that may be porous) or an adhesive (a glue) that may be porous. As discussed further below, when joined, a layer of intervening material (e.g., a porous polymer or porous fluoropolymer) may be placed between the flat faces of the D shaped lumens (see, e.g., FIG. 2, item 13).

When liners are joined together within a tube, an intervening layer of material may be located between the liners (that may act as a spacer between the liners). The intervening layer of material between the liners may be nonporous or porous. The intervening layer of material may have a density in a range selected from, for example, about 0.3 to about 1.9 g/cc. Exemplary densities of the intervening layer of material include from about 0.3 to about 0.9 g/cc. or from about 0.4 to about 0.9 g/cc. Exemplary densities of the intervening layer of material include from about 0.3 to 0. about 4 g/cc or from about 0.4 to about 0.5 g/cc. Exemplary densities of the intervening layer of material include from about 0.5 to about 0.6 g/cc or from about 0.6 to about 0.7 g/cc. Exemplary densities of the intervening layer of material include from about 0.7 to about 0.8 g/cc or from about 0.8 to about 0.9 g/cc. Exemplary densities of the intervening layer of material include from about 0.9 to about 1.4 g/cc, or from about 0.9 to about 1.9 g/cc. The intervening layer of material between the liners in those density ranges may be an expanded or electrospun polymer. The expanded or electrospun polymer may be, for example, PTFE (e.g., cPTFE) or ePVDF.

Catheters (e.g., HDCs) and other medical devices encompassed by the present disclosure include those having DD dual lumen structures in which the liners of the individual inner lumens 11 are made out of nonporous fluoropolymers and separated by an optional layer of porous fluoropolymer (e.g., PTFE or PVDF). See, e.g., FIG. 2 item 13.

Where multi-lumen tubing (e.g., dual-lumen, triple-lumen, or quadruple-lumen tubing) comprises an expanded fluoropolymer extruded with the outer wall and septa separating the lumens formed in the same extrusion process, the septa separating the lumens of the tubing may be comprised of the same or substantially the same porous fluoropolymer as the outer wall of the tubing. Any one or more (e.g., two or more) of the lumens may be lined by a nonporous liner. Accordingly, medical devices (e.g., dual- or triple-lumen catheters, cannulas, shunts and drains) may comprise tubing where the septa are formed from porous (e.g., expanded) fluoropolymers, including the same or substantially the same fluoropolymers as the outer wall of the medical device. Any one or more of the lumens may subsequently be lined using the technique described in FIG. 3.

The tubing described herein, and sections of the tubing described herein incorporated into medical devices (e.g., catheters, cannulas, shunts, drains, etc.), including the dual lumen catheters (e.g., dual lumen HDCs) of the present disclosure with a DD structure, may have liners within the lumens. The liners may be made out of nonporous polymers, fluoropolymers, and/or perfluoropolymers that are separated (spaced apart; see FIG. 2) by an intervening layer that may act as a spacer comprised of, for example, a porous fluoropolymer. The intervening layer/spacer may be inserted when, for example, forming the inner liner assembly 12, shown in FIG. 2, that is ultimately covered by a porous fluoropolymer coating. The intervening material/spacer may act as an adherent material; alternatively, a separate adherent material (e.g., adhesive) may be used. When the intervening layer of material/spacer is a porous fluoropolymer it may, for example, have a density in a range selected from about 0.3 to about 2.2 g/cc, about 0.3 to about 0.4 g/cc, about 0.4 to about 0.5 g/cc, about 0.5 to about 0.6 g/cc, about 0.6 to about 0.7 g/cc, about 0.7 to about 0.8 g/cc, about 0.8 to about 0.9 g/cc, about 0.9 to about 1.0 g/cc, about 1.0 to about 1.2 g/cc, about 1.2 to about 1.4 g/cc, about 1.4 to about 1.6 g/cc, about 1.6 to about 1.8 g/cc, about 1.8 to about 2.0 g/cc, or about 2.0 to about 2.2 g/cc. The porous spacer may, for example, comprise PTFE or PVDF, and have a density less than the density of nonporous PTFE or PVDF, respectively.

The rigidity of tubing or medical devices (e.g., catheters, shunts, cannulas and drains) is important in its ability to be inserted and maintained in a patient without damage to the device or damage to the patient's tissues, particularly when the area where the device (e.g., catheter, cannula, shunt, or drain) is inserted is mobile (e.g., near or passing through a joint that can bent or flex) or where the device must be surgically tunneled into final position. The material and/or thicknesses of each component of a tube (e.g., catheter tube) including the material and/or thickness of the outer wall, the inner liner(s), intervening material between the liners, and any adherent material joining them may be selected to control the longitudinal or torsional rigidity of the tubing (e.g., tubing used in a catheter). Changing the outer wall thickness or increasing the thickness or rigidity of liner sections adjacent to the outer wall 22 of the tubing will tend to increase the tortional rigidity of the tubing to a greater degree than its longitudinal rigidity. In contrast, increases in the thickness or rigidity of nonporous septa, or liner sections adjacent to the septa, will tend to have a greater effect on longitudinal than tortional rigidity.

Using the septa separating the lumens of tubing as reservoirs, as opposed to creating a thicker outer wall or adding an additional layer of porous material to the outer wall to increase its fluorinated liquid reservoir capacity, avoids substantial alterations (e.g., increasing) of the stiffness (torsionally or longitudinally) of the tubing that would result from a thicker outer wall or added layer that functions as a fluorinated fluid reservoir. In this manner, lined or unlined tubing (e.g., FIG. 4, structures (a) to (d)) may be provided with substantially the same fluorinated liquid reservoir capacity as a lined tube lacking porous septa (see, e.g., FIG. 4, structure (h)) without the need to increase wall thickness that potentially sacrifices longitudinal or tortional flexibility (e.g., relative to otherwise identical tubing). This permits the tubing to be more readily used in demanding applications such as medical devices where flexibility may be required. For example, a section of unlined dual-lumen tubing of the present disclosure made of ePTFE 5 mm diameter, with a wall thickness approximately 0.64 mm, and septum thickness approximately 0.9 mm may be wrapped around a 0.7 cm cylindrical rod without the lumens collapsing (e.g., kinking to substantially limit the lumen's cross section by, for example, more than 50%). By way of example, tubing of the present disclosure from about 6 to about 16 French (Fr). may be wrapped around a cylindrical rod of 0.7 cm without collapsing (kinking).

Alternatively, or in addition to controlling the outer wall, liner(s), adherent materials, etc., other methods of controlling the rigidity of the tubing and devices into which the tubing disclosed herein is incorporated (e.g., catheters, shunts, etc.) include the incorporation of metal wires and or plastic rods. For example, wire or rods may be included within the outer walls or septa of tubing used in a medical device such as a catheter. The rods or wires may be placed parallel with the lumens along all or part of the tube's length.

### 4.2.1 Dimensions

The outer diameter of the tubing, the thicknesses of the outer wall and septa between lumens, and the thickness of liners employed in the tubing may be separately controlled. The tubing may be greater than or equal to about 1 cm in diameter with the septa and/or wall thickness from less than about 2 mm (e.g., about 1 to about 2 mm). The diameter of the tubing may be less than or equal to about 1 cm and the septa and/or wall thickness less than about 1.5 or less than about 1 mm. The diameter of the tubing may be less than or equal to about 1 cm (e.g., from about 0.5 to about 1 cm) and the septa and/or wall thickness less than about 1 or less than 0.75 mm. The diameter of the tubing may be less than or equal to 0.5 cm (e.g., from 0.25 to 0.5 cm) and the septa and/or wall thickness less than 0.08 mm or less than 0.05 mm.

Sections of tubing used for medical devices, and accordingly the medical devices, may have dimensions in any of the above recited ranges. Additional ranges for sections of tubing described herein comprising porous (e.g., expanded) fluoropolymer walls when used for medical devices may be expressed using the French scale or French gauge system. The outer diameter of tubing of the present disclosure used in medical devices (e.g., catheters, cannulas, shunts, drains, etc.) may be, for example, from about 12 to about 30 French (Fr). Such tubing may be, for example, about 12 Fr (4 mm), about 12.5 Fr, about 13 Fr (4.333 mm), about 13.5 Fr, about 14 Fr (4.667 mm), about 14.5 Fr, about 15 Fr (5 mm), about 16 Fr (5.333 mm), about 18 Fr (6 mm), about 20 Fr (6.667 mm), about 22 Fr (7.333 mm), about 24 Fr (8 mm), about 26 Fr (8.667 mm), about 28 Fr (9.333 mm), or about 30 Fr (10 mm). For example, where the outer layer of a medical device (e.g., a catheter) is made of tubing comprised of porous fluoropolymer (e.g., a porous ePVDF or ePTFE), the device may have a diameter in a range selected from about 12 Fr to about 20 Fr or from about 20 Fr to about 30 Fr. The section of tubing in the device may also have a diameter in a range from about 12 Fr to about 16 Fr, or from 16 Fr to about 20 Fr. The section of tubing in the device may also have a diameter in a range from about 20 Fr to about 25 Fr, or from about 25 Fr to about 30 Fr. Alternately, the tubing may also be smaller, ranging from about 4 Fr to about 11 Fr, such as from about 4 Fr to about 6 Fr, from about 6 Fr to about 8 Fr, from about 8 Fr to about 10 Fr, or from about 10 Fr to about 11 Fr. For measurements given on the Fr scale, the term “about” is understood to mean+/−0.75 Fr units (as 3 Fr units=1 mm, “about” may be understood to mean+/−0.25 mm). Fractional Fr units expressed in mm above are rounded at the third decimal place.

For example, a catheter or other medical device may have a size selected from about 4 Fr to about 11 Fr or from about 12 Fr to about 16 Fr. A catheter or other medical device may have a size selected from about 4 Fr, about 5 Fr, about 6 Fr, about 7 Fr, about 8 Fr, about 9 Fr, about 10 Fr, about 11 Fr, about 12 Fr, about 12.5 Fr, about 13 Fr, about 13.5 Fr, about 14 Fr about 14.5 Fr, about 15 Fr, about 15.5 Fr., or about 16 Fr.

The thickness of the outer walls (the thickness of the thinnest part of each wall between a lumen and the exterior of the tubing section) and the thickness of each septa between lumens (e.g., the thinnest part of the septa) of tubing used for non-medical and medical purposes (e.g., catheters, cannulas, drains, or shunts) may be independently selected. Each wall thickness and each septa thickness may be independently selected and may, for example, be in a range from about 5% to about 20% of the shortest cross-sectional length perpendicular to the longitudinal axis of the tubing (e.g., the diameter where the tube has the smallest circular cross-section). Each wall thickness and each septa thickness is independently selected and may be, for example, in a range from about 5% to about 10% or from about 10% to about 20% of the shortest cross-sectional length perpendicular to the longitudinal axis of the tubing (e.g., the diameter where the tube has the smallest circular cross section). For example, for tubing employed in a catheter or other medical device from about 13 Fr (4.333 mm) to about 22 Fr (7.333 mm), the wall thickness may be about 0.45 mm to about 0.8 mm thick and the septa thickness (e.g., for a dual lumen catheter) may be from about 0.6 to about 1.1 mm thick.

Any one or more of the lumens in tubing used for medical and non-medical purposes may be unlined or lined (e.g., with a solid (nonporous) polymer, fluoropolymer, or perfluoropolymer). When lined, the type of material used for the liner and the thickness of the liner may be selected independently for each lumen. Suitable liner thicknesses may be, for example in the range of about 10 to about 160 microns. Suitable ranges for liner thickness include, but are not limited to, 10-20 microns or 20-40 microns. Suitable ranges for liner thickness include, but are not limited to, 40-60 microns or 60-80 microns. Suitable ranges for liner thickness include, but are not limited to, 80-100 microns or 100-120 microns. Suitable ranges for liner thickness include, but are not limited to, 120-140 microns or 140-160 microns.

Where a medical device such as a catheter comprises tubing of the present disclosure (e.g., catheters having an overall DD structure including HDCs) having a porous fluoropolymer outer layer over all or part of its surface (e.g., the part intended to be inserted into a patient), nonporous fluoropolymer liners may be present in one, two, or more of the lumens of the catheter.

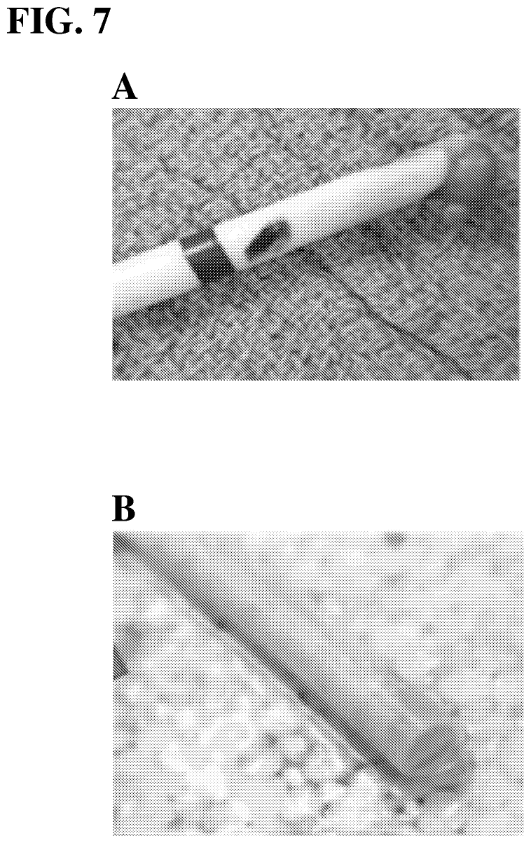

The end of a medical device (e.g., the portion of a catheter, cannula, or drain intended to be inserted into a patient) formed from a section of tubing described herein may be tapered from a diameter equal to the diameter of the tubing to a narrower diameter or even a point at its tip to facilitate its placement. The end of the section of tubing may also have holes in it. The holes may be in the tube wall (i.e., placing at least one lumen and the exterior of the tube in fluid communication via the hole) and/or place two or more lumens in fluid communication with the area exterior to the tube (e.g., the area within an artery or vein in which a catheter is implanted). The distal end and/or tip of a medical device, such as the end of a catheter or cannula, that is inserted into a patient may have a hole in it (a hole at the tip, sec, e.g., FIG. 7B); that is to say, the catheter's tip ends not in a closed solid structure, but in a hole placing at least one of the lumens in fluid communication with the exterior. When a hole is placed at the tip of a device, such as a catheter, the hole may be less than the diameter of at least one of the lumens, or less than the diameter of the smallest lumen at the point where the hole forms a connection between the lumen and the exterior of the catheter. The hole at the tip may also be formed by cutting the tubing used to form the device in a plane perpendicular to the longitudinal axis of the tubing or at one or more angles oblique to the longitudinal axis of the tubing used to form the device.

### 4.2.2 Reservoirs

Two forms of reservoirs for holding fluorinated liquids are provided in the tubing described herein and, accordingly, the medical devices into which the tubing is incorporated. Both forms of reservoirs permit fluorinated liquid(s) stored in the reservoirs to reach the surface of the porous (e.g., expanded) fluoropolymers and combine with it to form a slippery surface that can be biopassive and resistant to fibrin sheath and/or thrombus formation when used in medical applications.

The first form of reservoir is the pores of porous fluoropolymers (e.g., ePTFE) used to form the septa (walls) between the lumens exemplified in the cross sections shown in FIG. 4. Using the septa 23 as a reservoir avoids having to add an additional layer of porous material to the wall of the tubing thus increasing its thickness and/or the diameter of the tubing. As indicated above, using the septa 23 or 25 as a reservoir also avoids increasing, for example, the stiffness of the tubing (e.g., torsional and/or longitudinal stiffness) relative to a tube that does not have material added to the wall to act as a reservoir. Fluorinated fluid in the septa can migrate (e.g., via the pores) to the outer wall 22 of the tubing. Although the septa do not uniformly contact all parts of the outer wall equally, and in some cases contact only discrete sections of the outer wall (see, e.g., the dual-lumen catheters in FIGS. 2, 3, and 4, structures (a) to (g)), the septa act as a reservoir for the entire surface of the tubing or device into which it is incorporated, permitting the entire surface to remain slippery. That results in blocking of, for example, formation of fibrin sheathing and thrombi on the surface of the tubing when implanted as all or part of a device into a mammal (e.g., a vein of the vascular system). FIG. 4 illustrates the flow of fluorinated liquid in the cross sections shown at (a) to (d) by the solid arrows extending from the septa to the outer wall. The tubing may be unlined as in (a) and (b) or lined with a liner 24 of nonporous material as in (c) and (d). Cross sections shown in (a) and (c) comprise septa of substantially uniform thickness as opposed to the septa 25 in cross sections (b) and (d) having a cross section that become thicker as it approaches the outer wall 22. Porous septa that become thicker near the outer wall permit the fluid in the reservoir to be in closer proximity to the outer wall, and can permit the tube to have lumens of larger cross sectional area. Two variations of dual lumen tubes with porous septa and optionally lined lumens of unequal size are provided at (e) and (f). A triple lumen tube with porous septa dividing the lumens of unequal size is provide at (g). The lumens may be made equal in size and optionally any one, two or three of the lumens are lined.

As indicated above, the septa and walls of tubing used for non-medical and medical purposes may be of nonuniform thickness. For instance, the thinnest part of at least one septa in a tube may be less thick than the thinnest part of the tube wall. Alternatively, the thinnest part of at least one septa in a tube may be thicker than the thinnest part of the tube wall. In addition, the porosity of the material used to form the septa need not be the same as the material used to form the outer wall, even where the materials are chemically identical (e.g., ePTFEs of different densities may be used).

The second form of reservoir is a lumen of the tubing (luminal space or reservoir lumen) that is filled with fluorinated liquid. An embodiment of the second form of reservoir is an unlined lumen formed in the porous polymer of the tubing (see, e.g., item 26 in structure (g) of FIG. 4). Another embodiment of the second form of reservoir is a luminal space 29 formed between the outer wall 22, and an infold in a nonporous liner 28. Examples of luminal spaces formed by an infold of the liner are depicted in FIG. 4, structures (i) through (n), where one infold (see structures (k) and (l)) or two infolds (see structures (i), (j), (m), or (n)) are employed to form luminal reservoirs in the presence of a porous septa or a nonporous septa.

Open ended lumens may be utilized as reservoirs with the fluorinated liquid retained in the reservoir by affinity for the environment in the lumen (e.g., interaction with the porous fluorinated polymer of the tubing). Either one or both ends of a tube's lumen may be narrowed to a very small opening (e.g., a pore) and/or fitted with a valve or valve like structure that resists the flow of fluid but will permit passage with sufficient pressure. One such example is a small slit in the tubing wall or a one way valve. A reservoir lumen may be sealed (e.g., blocked or closed by a septum that permits access by, for example, a needle). For example, where a multi-lumen section of tubing is employed as a medical device such as a catheter or cannula, the lumen serving as a reservoir (e.g., lumen 26 of structure (g) in FIG. 4) may be sealed off in the distal portion (e.g., at the tip) inserted into the patient. Alternatively, the portion inserted into the patient may restrict fluid outflow by being narrowed in size with an opening at the tip less than 10% or less than 5% of the lumen's cross-sectional area. Another alternative is to place a one-way valve or a small slit (e.g., less than 1.5 mm in length) in the tube wall at or near the implanted end (in the implanted portion). The slit or valve provides fluid communication between the exterior of the tube and the lumen used as a reservoir. Pressure may force fluid through the slit but it otherwise remains sealed. The other end of the lumen (e.g., the proximal end of the catheter or cannula that remains external to a patient when the distal end of a catheter or cannula is inserted) can be sealed off or closed with a valve or septum that permits access to the reservoir so that, for example, fluorinated fluid can be added or removed. When the distal end is open, as with a narrow opening, slit, or one-way valve, fluorinated liquid can be added to the reservoir lumen without a buildup of excess pressure in the lumen, while still substantially retaining the fluorinated fluid in the reservoir. Where medical devices comprising a reservoir to hold fluorinated liquid are fully implanted, both ends of the lumen forming the reservoir space may be closed off. Generally, the luminal space used as a reservoir for fluorinated fluid will be formed in the porous (e.g., expanded) fluoropolymer so the fluorinated fluid in the reservoir can move into the pores and replenish fluorinated liquid at the surface of the tube (e.g., the surface of the tube that will form a catheter or cannula). The luminal space can be formed completely in an unlined porous fluoropolymer (see, e.g., item 26 in FIG. 4, structure (h)) or, where part of the luminal space is lined (formed by), with a nonporous liner (see, e.g., lumina space 29 formed by an infold of liner 28 in FIG. 4, structures (i) to (n)).

It is also possible to replenish the fluorinated liquid in the tubing, particularly the sections of tubing incorporated into a medical device that has been implanted into a patient (e.g., a HDC), by filling one or more of the lumens of the tubing with a fluorinated liquid during periods where flow through the one or more lumens is not required for operation. For example, filling one or more lumens of a medical device with locking solution comprising the fluorinated liquid and locking off the accessible (generally external) portion of the device. Unlike using a device with a lumen functioning as a dedicated reservoir, replenishing the tubing/device with fluorinated liquid in this manner allows the use of fewer lumens and accordingly, the remaining lumens can be of larger cross section for any given size and shape of tubing. Replenishing the fluorinated liquid in this manner is, however, subject to loss of the liquid through the open end of a lumen or any other opening in the tubing (e.g., openings in the distal tip of an implanted section of a catheter or other medical device).

Where a medical device comprising tubing of the present disclosure is totally implanted (e.g., as in a hydrocephalus shunt), neither the distal nor proximal ends of the devices are directly accessible. The device may be replenished with fluorinated liquid by connecting a reservoir lumen of the device with a transcutaneous access port.

Where no other means of replenishing the fluorinated liquid of an implanted device is available, perfluorinated liquids such as perfluorodecalin may be administered to the patient (e.g., intravenously). A portion of the fluorinated liquid will become associated with the porous fluorinated polymer.

### 4.2.3 Marking and Contrast Agent Incorporation

Radiopaque markers and/or contrast agents may be incorporated into any of the polymers used to prepare the tubing, particularly tubing for use in the preparation of medical devices such as catheters, cannulas, drains, or shunts, to aid in placement of the device. Such materials permit devices such as catheters to be imaged during and after catheter placement (implantation) in a patient using various X-ray technologies including, but not limited to, X-ray images, Computer Assisted Tomography (CT) and fluorography.

The incorporation of radiopaque materials may be accomplished in a variety ways. For example, the radiopaque markers may be incorporated in the polymers used to manufacture the porous wall/coating of the tubing and/or one or more (e.g., two or more) of the polymers used to manufacture liners. Where wires or rods are provided in the catheter to control its rigidity, the wires or rods when made of metal may serve as a radiopaque marker. Alternatively, where the wire or rod is comprised of a rigid plastic or polymer (e.g., a length of rigid engineered polymer rod), the plastic or polymer may function as or include a radiopaque material. Another way of incorporating radiopaque markers is the impregnation of radiopaque materials in the porous polymer along the length of the catheter (e.g., as dots of radiopaque material a distance below the surface of the porous polymer). Radiopaque inks may also be printed (e.g., pad printed) onto the catheter. In such a process the ink preferably infiltrates the pores of the ePTFE before the ink dries (e.g. air cures). Such a printing process can produce imbedded contrast agent on catheters that resists removal with isopropyl alcohol, or with adhesive tape (e.g., in a manner similar to ASTM D3359). Injecting drops of contrast agent within the bulk of the catheter will produce similar results. Placement of radiopaque markers may be limited to the tip of the catheter.

As an alternative, or in addition, to placing radiopaque markers in or on the polymers of the catheter, a contrast agent can be used to fill one or more of the catheter's lumens before, during or after implantation to improve the radiopacity of the catheter (e.g., a medical device's radiopacity in a mammal in vivo).

### 4.3 Processes of Preparing Tubing and Medical Devices

Tubing of the present disclosure, and medical devices (e.g., catheters, cannulas, drains, or shunts) that comprise a section of tubing of the disclosure may be prepared by a variety of processes. A first method for preparing lined tubing begins with the fabrication of a liner (e.g., a dual-, triple- or quadruple lumen liner) by extrusion, or as an inner liner assembly (see, e.g., item 12 in FIG. 2). Subsequently, a layer of porous fluoropolymer is applied to the surface of the liner by extrusion or by inserting the liner into a tube of porous fluoropolymer. For example, an extruded polyurethane tube or polyurethane inner liner assembly may be inserted into an ePTFE sleeve. The liner may be sintered to the fluoropolymer as necessary. A second method of preparing tubing of the present disclosure is extrusion forming of a porous (e.g., expanded) fluoropolymer tube (e.g., a dual-, triple- or quadruple-lumen tube). A third method of forming tubing of the present disclosure is the insertion of liners into one, two, or more lumens of a porous fluoropolymer tube (e.g., prepared by extrusion in the second method), for example, as shown in FIG. 3 and discussed below. Once the tubing itself is assembled, it is treated with a fluorinated (e.g., perfluorinated liquid) such as perfluorodecalin to form a slippery surface that resists fouling (e.g., biological fouling including fibrin sheath and thrombus formation when implanted in a mammalian vein). Treatment with a fluorinated liquid may occur immediately after the tubing is formed, after a section of tubing becomes part of a non-medical or medical device, or at any time prior to use. In instances where the fluorinated liquid can reach the surface of the tubing, the tubing may be installed (e.g., a medical device implanted) and then treated with the fluorinated liquid in position (i.e., in situ).

The first method of preparing tubing of the present disclosure for use in non-medical and medical applications is exemplified in FIG. 2 where liners for the lumen of the tubing are shaped and assembled into an inner liner assembly 12 that is subsequently covered with a porous (e.g., expanded) fluoropolymer. For example, when preparing catheters having a DD structure with both lumens having an inner liner made out of nonporous polymers or fluoropolymers and an outer layer made out of porous fluoropolymer, each of the D-shaped liners can be produced by extrusion processing and shaped on a mandrel if necessary. The liners are then brought together with an optional intervening layer of material (e.g., porous fluoropolymer and/or adherent material between them), after which a porous fluoropolymer is applied over them either by extrusion forming or by sliding the inner liner assembly into a porous polymer tube. Once the desired portion of the inner liner assembly is covered with the porous fluoropolymer, the entire assembly may be sintered together.

More specifically, and with reference to FIG. 2, in the first method the liners 10 are formed into D-shaped inner liners 11 by passage over D-shaped mandrels. Two D-shaped liners are joined together to form an inner liner assembly (structure below the bracket at 12) using an optional intervening layer of material 13 e.g., an adherent material, such as an adherent porous or nonporous fluoropolymer, or a polymeric material that can withstand any subsequent sintering temperature. Sintering of the inner liner assembly, if desired (e.g., for bonding the liner and adherent material components), may be conducted at a temperature ranging from 200-300° C. 300-400° C. or 400-500° C. depending on the polymers employed. Once formed, all or part (e.g., the part intended for insertion into a patient) of the inner liner assembly (e.g., DD inner liner assembly 12 of FIG. 2) is covered with a porous fluoropolymer exterior layer 14 (e.g., ePTFE coating by extrusion processing or by insertion into a porous ePTFE tube), and the entire assembly is optionally sintered together forming, among other things, the outer wall 15, a portion of which is circled by a dashed line in FIG. 2. A cross section of a double lumen catheter through a portion coated with porous fluoropolymer 14 after optional sintering is shown to the left of the bracket at 16 in FIG. 2. Where no layer of adherent material, such as a porous fluoropolymer, is located between the faces of the liners used to form the inner liner assembly, the inner liner assembly may be replaced by an extruded nonporous dual-lumen tube. The components of the assembly are held in place by a variety of mechanisms including, but not limited to, compression, friction, and/or bonding (e.g., bonding between thermoplastics or adhesives and catheter components). Although adhesives and other thermoplastics do not tend to bond well to fluoropolymers such as FEP and PTFE, the exterior layer of porous fluoropolymers (e.g., ePTFE) is porous and, in addition to compressive and frictional forces holding the coating in place, the adhesives and/or thermoplastics may intercalate into the pores (e.g., a portion of the pores) of the porous material, providing additional bonding. The technique used to prepare double walled tubing, including tubing used in medical applications and devices such as catheters and cannulas, may be used to prepare tubing with more lumens (e.g., triple- and quadruple-lumen tubing) by adjusting the size and shape of the mandrels used to form each liner, which may be selected independently.

The second method of preparing tubing, including tubing for the preparation of catheters and other medical devices of the present disclosure, is extrusion forming the catheter out of porous (e.g., expanded) fluoropolymer or perfluoropolymer. Extrusion forming permits the septa of the catheter to be prepared from fluoropolymers that differ in chemical composition and/or porosity from the porous (e.g., expanded) fluoropolymer used to prepare the outer wall. Extrusion formed tubing may have one or more lumens separated by septa with a uniform thickness between lumens (see, e.g., FIG. 4 at a or c) or by septa where the thickness of the septa between any two lumens varies. FIG. 4 at b and d exemplifies a septum between two lumens of varying thickness, with the thickness of a portion of the septum (e.g., in the center of the tube) being thinner than the thickness of the septum closer to the outer wall of the tube. The thinnest portion of the septum may also be thicker than the thinnest portion of the outer wall of the tube.

The third method of forming tubing, including tubing for the preparation of catheters and other medical devices of the present disclosure, comprises adding a liner to a porous (e.g., expanded) fluoropolymer tube, such as a tube prepared by the second method. Each inner liner can be added by first loading the liner on an appropriately shaped mandrel (e.g., a D-shaped mandrel for a dual lumen tube). Liners may be inserted into two or more lumens of a tube simultaneously as shown for the preparation of a lined dual-lumen tube in FIG. 3. Before sliding the porous fluoropolymer/perfluoro-polymer over the liner and mandrel assembly, a fluoropolymer (e.g., FEP or PTFE) dispersion in water can be sprayed over the liner to enhance the adhesion between the liner and the porous fluoropolymer layer upon sintering.

Any of the fluoropolymers recited above may be employed as porous (e.g., expanded) fluoropolymers for the preparation of the tubing by the above-mentioned methods and, accordingly, for the preparation of any non-medical or medical devices comprising the tubing. In particular, expanded PTFE may be employed. For example, ePTFE with a density of about 1.0 g/cc to about 1.4 g/cc or about 1.1 g/cc to about 1.3 g/cc may be employed to prepare tubing, non-medical devices, or medical devices of the present disclosure. ePTFE having a density of 1.2+/−0.05 g/cc falls in both of those ranges.

Tubing of the present disclosure may be incorporated into medical devices that are partially or completely inserted or implanted into a patient (e.g., a mammalian patient). The portions of the medical devices, such as catheters, that are not intended to be inserted into a patient/subject (e.g., sections that may not be coated with a porous fluoropolymer exterior layer) may be coated (e.g., clad, wrapped, encased, or otherwise covered) with a protective layer of material. The material may be textured and/or shaped to provide a better grip. The material may be, for example, a thermoplastic applied as a polymer sleeve that is “shrink wrapped” into place. Coatings on this portion of the catheter may be applied after any sintering of the porous fluoropolymer into place, or before any sintering so as to bond the coating and the porous fluoropolymer to any liners or an inner liner assembly as indicated above. The coating provided for protection of the liner assembly and/or grip may overlap with the porous fluoropolymer coating and is not shown in the figures (e.g., not shown in FIG. 1 at B).

### 4.4 Packaging and Sterilization

Tubing, non-medical devices, and medical devices (e.g., catheters such a HDCs) made with porous (e.g., expanded) fluoropolymer or porous perfluoropolymer once formed are required to be infused with one or more fluorinated liquids (described above) in order for tubing, non-medical devices, or medical devices (e.g., catheters) to resist fibrin sheath formation, thrombus deposition, and/or pathogen colonization in vivo. Prior to being infused with the fluorinated liquid, the tubing, non-medical devices, or medical devices (e.g., catheters) may be sterilized (e.g., with ethylene oxide) then pre-sterilized fluorinated liquid (e.g., fluorocarbon and/or perfluorocarbon liquid) is dispensed into the packaging before sealing the package from liquid leakage and/or evaporation.

Fluorinated liquid (e.g., fluorocarbon and/or perfluorocarbons such as perfluorodecalin) contacted with the tubing, non-medical devices, or medical devices (e.g., catheters) can be sterilized before it is infused into the tubing, non-medical devices, or medical devices (e.g., catheters) by, for example, filter sterilization (e.g., with a 0.22 micron filter). Infusion can be conducted by contacting the fluorinated liquid with the porous fluoropolymers of the tubing, non-medical devices, or medical devices (e.g., the whole catheter or the portions prepared from porous fluoropolymers). As indicated above, the infusion may be conducted at the time of manufacture and the infused tubing, non-medical device, or medical device (e.g., catheter) packaged. Alternatively, the infusion step can be conducted at the point of care using an aliquot of sterile fluorinated liquid. Where the device comprises a lumen used as a reservoir to hold the fluorinated liquid, it may be filled prior to packaging or at any other appropriate time prior to use. Medical devices that comprise such a reservoir and can be accessed after implantation (e.g., as in the case of a catheter and shunt) can have the reservoir filled or refilled at any time before, during, or after implantation of the device.

### 4.5 Applications

The tubing described herein, including dual-, triple, quadruple-lumen, and other multiple lumen tubing has a number of properties that make it useful in an array of applications. The tubing may be used as a medical device or incorporated into medical devices. In some instance the tubing is used external to a patient (e.g., tubing for the external portion of hemodialysis or extracorporeal membrane oxygenation (ECMO). In other instances, the tubing may be part of a device that is fully or partially implanted into a patient such as catheters, cannulas, drains, or shunts.

In some instances, the medical devices (e.g., catheters, such as HDCs) described herein are inserted fully or partially into the body of a patient (e.g., into a vein or artery) and permit access to internal portions of a patient's or subject's body, including for the introduction and/or withdrawal of fluids (e.g., blood, cerebrospinal fluid, lymph etc.) and other bodily substances. Because of its resistance to fibrin sheath and thrombus formation for an extended period of time, the tubing finds use, for example, in devices such as catheters for hemodialysis and other applications where an indwelling line is implanted into the vasculature (e.g., blood stream) for an extended period of time. While the tubing resists fibrin sheath formation and the formation of thrombi, the wall of the vessel into which the catheter is inserted may seal to the catheter (e.g. the seal may be made to the porous fluoropolymer tubing section of the catheter or to a section of other tubing attached thereto) providing a stable implant that does not lose blood or plasma. The tubing also resists the attachment of other mammalian cell types and accordingly may be used for implantation outside of the blood circulation system.

The tubing described herein may also be utilized in medical drains and shunts. In some instances, the tubing is used to prepare all or part of a shunt. The shunt may be used to treat hydrocephalus (a hydrocephalus shunt) with an end (e.g., the tip) implanted (fitted) in a ventricle of the brain or subarachnoid space, where the catheter resists the attachment of cells (e.g., glial cells such as astrocytes and inflammatory cells) that can bind to and block the shunt, requiring its replacement. Near the tip of the hydrocephalus shunt numerous holes may be formed in the side of the tubing used to form the shunt (e.g., more than 10, more than 20, or more than 30 holes) to permit drainage of fluid (e.g., cerebrospinal fluid). Shunts may comprise more than one lumen (e.g., a dual-lumen or triple lumen shunt) such that septa between two or more lumens may be used as a reservoir for the fluorinated liquid. Shunts may also comprise a lumen that is filled with and acts as a reservoir of fluorinated liquid (e.g., one lumen of a dual lumen shunt may be filled with fluorinated liquid and sealed before implantation). Shunts with a septa and/or filled lumen reservoir will provide a slippery surface that resists cell adhesion and/or blockage for a period of time greater than shunts (e.g., hydrocephalus shunts) that do not have a slippery surface, or shunts that have a slippery surface but do not have a reservoir of fluorinated liquid.

The tubing may repel water and may in some instances be hydrophobic or omniphobic. Ommiphobic materials are both hydrophobic and oleophobic. The surface of tubing described herein may be water repellant with a water roll off angle (angle of incline a 50 microliter water drop will roll off a substantially planar surface) of less than 10° or less than 5° at 22° C. When incorporated into a medical device, sections of tubing of the present disclosure implanted into mammalian vasculature (e.g., a vein) may retain a roll off angle less than 10° or less than 5° after 30 or 60 days of implantation. When incorporated into a medical device, sections of tubing of the present disclosure implanted into mammalian vasculature (e.g., a vein) may retain a roll off angle less than 10° or less than 5° after 90 days of implantation.

The surface of tubing of the present disclosure may have a static contact angle with water that is greater than about 70° or greater than about 90° at 22° C. When incorporated into a medical device, sections of tubing of the present disclosure implanted into mammalian vasculature (e.g., a vein) may retain a water contact angle greater than 70° or greater than 90° after 30 days of implantation. When incorporated into a medical device, sections of tubing of the present disclosure implanted into mammalian vasculature (e.g., a vein) may retain a water contact angle greater than 70° or greater than 90° after 60 days of implantation. When incorporated into a medical device, sections of tubing of the present disclosure implanted into mammalian vasculature (e.g., a vein) may retain a water contact angle greater than 70° or greater than 90° after 90 days of implantation. Such contact angles may be measured with perfluorodecalin as the fluorinated fluid associated with the tubing. Water contact angles are measured in air using a goniometer (e.g., an Attension Model Theta goniometer, available from BIOLIN SCIENTIFIC, formerly KSV Instruments, Stockholm, Sweden). For the purposes of this disclosure, the surface of a material is hydrophobic if it has a static contact angle with water greater than 90° at 22° C. and one atmosphere of air pressure.

For the purposes of this disclosure, an oleophobic material or surface is one that results in a dodecane droplet forming a static surface contact angle exceeding about 90° at 22° C. at one atmosphere in air measured using a goniometer (e.g., Attension Model Theta goniometer, formerly KSV Instruments, available from BIOLIN SCIENTIFIC, Stockholm, Sweden). When incorporated into a medical device, sections of tubing of the present disclosure implanted into mammalian vasculature (e.g., a vein) may retain a dodecane contact angle greater than 60° or greater than 90° after 30 days of implantation. When incorporated into a medical device, sections of tubing of the present disclosure implanted into mammalian vasculature (e.g., a vein) may retain a dodecane contact angle greater than 60° or greater than 90° after 60 days of implantation. When incorporated into a medical device, sections of tubing of the present disclosure implanted into mammalian vasculature (e.g., a vein) may retain a dodecane contact angle greater than 60° or greater than 90° after 90 days of implantation. Such contact angles may be measured with perfluorodecalin as the fluorinated fluid associated with the tubing.

As discussed above, medical devices such as catheters (e.g., HD catheters), cannulas, shunts, drains, etc., comprising a section of tubing described herein resist the formation of fibrin sheaths and/or thrombi when it is inserted into the vasculature (e.g., vein or artery) of a mammal. This is particularly the case where the section tubing, and accordingly the devices into which it is incorporated, comprises a reservoir in the form of a porous fluoropolymer septum and/or lumen used as a reservoir (lumen reservoir). The portion of a medical device comprising a section of tubing of the present disclosure that is implanted in an artery and/or vein of a mammal (e.g., the distal end of a catheter such as an HDC) may resist the formation of one or more thrombi and/or the formation of fibrin sheathing for at least 30 days or at least 60 days on the surface of the tubing. Such devices may resist the formation of one or more thrombi and/or the formation of a fibrin sheath on the section of tubing for at least 90 days. Less than 20% or 15% of the section of tubing implanted in the vasculature of a mammal (e.g., the portion of a catheter comprised of tubing of the present disclosure) may be covered by fibrin sheathing and/or thrombi for at least 30, at least 60 or at least 90 days. Less than 12% or 10% of the implanted section of tubing (e.g., the portion of a catheter comprised of tubing of the present disclosure) may be covered by fibrin sheathing and/or thrombi for at least 30, at least 60 or at least 90 days. Less than 8% or 6% of the implanted section of tubing (e.g., the portion of a catheter comprised of tubing of the present disclosure) may be covered by fibrin sheathing and/or thrombi for at least 30, at least 60 or at least 90 days.

In addition to resisting the formation of fibrin sheaths and thrombi when implanted in the vasculature of a mammal as part of a medical device, tubing of the present disclosure resists occlusion (blockage) of openings formed in its porous fluoropolymer walls. More specifically, openings in the tubing that permit fluid communication between one or more lumens of a device (e.g., a catheter) and the exterior space into which the device is inserted (e.g., the lumen of a vein) resist occlusion by fibrin sheath or thrombi formation. Where medical devices comprise a section of tubing described herein that have one or more openings in the tubing between the lumens and the exterior of the medical device, the openings remain substantially unoccluded (unblocked) even after extended periods of implantation in mammalian vasculature. When a medical device comprising tubing is implanted in other locations (e.g., cranially as a hydrocephalus shunt), the implanted tubing resists the adhesion of glial cells that may form a sheath or occlude the openings that permit fluid communication between a lumen of the device and its exterior (e.g., the drainage of cerebrospinal fluid by a hydrocephalus shunt). The opening in the implanted tubing may remain less than 40% or less than 30% occluded at 30 or 60 days post implantation relative to the size of the openings prior to implantation. The opening may remain less than 20% or less than 10% occluded at 30 or 60 days post implantation relative to the size of the openings prior to implantation. The opening may remain less than 8% or less than 5% occluded at 30 or 60 days post implantation relative to the size of the openings prior to implantation. The opening may remain less than 40% or less than 30% occluded at 90 days post implantation relative to the size of the openings prior to implantation. The opening may remain less than 20% or less than 10% occluded at 90 days post implantation relative to the size of the openings prior to implantation. The opening may remain less than 8% or less than 5% occluded at 90 days post implantation relative to the size of the openings prior to implantation.

For catheters employed in human hemodialysis (HDCs), a blood flow greater than about 350 milliliters per minute (ml/min) or 300 ml/min is considered desirable. When catheters that support flow rates above 350 ml/min when initially implanted fall below 350 or 300 ml/min the catheter is considered to be becoming occluded. HDCs having a distal end comprised of tubing of the disclosure can support flow rates of at least 350 ml/min or at least 300 ml/min when the distal end is implanted in an artery and/or vein of a mammal for extended periods. Such HDCs may support a flow rate of at least 350 ml/min for at least 30 days or at least 60 days post implantation. Such HDCs may support a flow rate of at least 350 ml/min for at least 90 days post implantation. Such HDCs may support a flow rate of at least 300 ml/min for at least 30 days or at least 60 days post implantation. Such HDCs may support a flow rate of at least 300 ml/min for at least 90 days post implantation.

The tubing of the present disclosure when incorporated into a medical device resists the formation of biofilms for extended periods when implanted into the vasculature of a mammal. The section of the device comprising tubing of the present disclosure may resist biofilm formation for at least 30 or at least 60 days as assessed by the amount of Staphylococcus epidermidis (colony forming units per cm of catheter surface area) that can be cultured from a section of implanted catheter after sonicating the section for 30 seconds in culture media. A section of the device comprising tubing of the present disclosure may resist biofilm formation for at least 90 days of implantation as assessed by the amount of Staphylococcus epidermidis that can be cultured from a section of implanted catheter after sonicating the section for 30 seconds in culture media.

Periodically, an aliquot of fluorinated liquid (e.g., perfluorodecalin or perfluorotributylamine) may be administered to a patient in which a medical device (e.g., a catheter, shunt, cannula, or drain) comprising a section of the tubing described herein has been implanted. The administered fluorinated liquid can replenish some or all of the fluorinated liquid that has been infused into the porous (e.g., expanded) fluoropolymers of the medical device. A fluorinated liquid can be administered through the implanted device if a portion of it is accessible when the device is implanted (e.g., the proximal portion of a catheter that remains external to a patient). For example, a fluorinated liquid may be incorporated into a locking solution used to fill one or more lumens of tubing incorporated into a medical device. Where a locking solution comprising a fluorinated liquid is used to fill all or part of an unlined porous fluoropolymer tube of the device, the fluorinated liquid will have direct access to the pores of the fluoropolymer on the inner surface of the lumen, and can enter the pores directly. Alternatively, because fluorinated liquids have preferential affinity for fluorinated materials, they can enter the patient or subject through the device and be circulated (e.g., in the blood), with some or all of the fluorinated liquid ultimately becoming associated with the porous fluoropolymer of the device. As discussed above, replenishment of fluorinated liquid associated with the porous fluoropolymer may also be accomplished by refilling a lumen of the tubing that functions as a reservoir either through a septum or valve that provides access to the lumen acting as a reservoir.

### 5.0 CERTAIN ASPECTS

1. Tubing comprising: (i) at least two lumens separated by septa (walls between the lumens), (ii) an outer wall having an outer surface located on the external face of the tubing, and (iii) a fluorinated liquid; wherein
the outer wall and the septa are comprised of a porous fluoropolymer having pores, and at least a portion of the fluorinated liquid is absorbed into the pores (of both the septa and the outer wall), and a portion of the fluorinated liquid is located (adsorbed) on the outer surface of the tubing (forming e.g., a slippery surface).

2. The tubing of aspect 1, wherein the pores of the porous fluoropolymer of the septa are in fluid communication with the pores of the porous fluoropolymer of the outer wall, and the fluorinated liquid in the septa can flow into the porous fluoropolymer of the outer wall and become located on the outer surface of the tubing. See, e.g., FIG. 4, structures (a) to (g), (i), and (j).

3. The tubing of any preceding aspect wherein the tubing comprises two septa separating three lumens (e.g., is triple lumen tubing). See, e.g., FIG. 4, structure (g).

4. The tubing of any preceding aspect, wherein the tubing comprises three or more septa separating four or more lumens (e.g., is quadruple lumen tubing) or four or more septa separating five or more lumens.

5. The tubing of any of aspects 3-4, wherein two or more of the septa are comprised of a porous fluoropolymer the pores of which are in fluid communication with the pores of the porous fluoropolymer of the outer wall. See, e.g., FIG. 4, structure (g).

6. The tubing of any of aspects 4-5, wherein three or more of the septa are comprised of a porous fluoropolymer the pores of which are in fluid communication with the pores of the porous fluoropolymer of the outer wall.

7. The tubing of any preceding aspect, wherein all of the septa separating lumens within the tubing are comprised of a porous fluoropolymer the pores of which are in fluid communication with the pores of the porous fluoropolymer of the outer wall. See, e.g., FIG. 4, structures (a) to (g), (i), and (j).

8. The tubing of any preceding aspect wherein one or more lumens is not lined with a nonporous liner. See, e.g., FIG. 4, structures (a), (b), and (g).

9. The tubing of any preceding aspect wherein none of the lumens are lined with a nonporous liner. See, e.g., FIG. 4 structures, (a), (b), and (g).

10. The tubing of any of aspects 1-8, wherein one or more of the lumens is lined with a nonporous liner. See, e.g., FIG. 4, structures (c) to (f), (i), and (j).

11. The tubing of any of aspects 3-8 wherein two or more of the lumens are lined with a nonporous liner.

12. The tubing of any of aspects 4-8 wherein three or more of the lumens are lined with a nonporous liner. See, e.g., FIG. 4, structures (c) to (f), (i), and (j).

13. The tubing of any of aspects 1-7, wherein all of the lumens are lined with a nonporous liner. See, e.g., FIG. 4, structures (c) to (f), (i), and (j).

14. Tubing comprising: (i) an outer wall having an outer surface located on the external face of the tubing, (ii) at least one nonporous liner within and contacting the outer wall, the liner comprising a lumen, and (iii) a fluorinated liquid; wherein
the outer wall is comprised of a porous fluoropolymer having pores, and
at least a portion of the fluorinated liquid is absorbed into the pores, and a portion of the fluorinated liquid is located (adsorbed) on the outer surface of the tubing (forming, e.g., a slippery surface). See, e.g., FIG. 4, structures (c) to (f) and (h) to (n).

15. The tubing of aspect 14, comprising two nonporous liners each comprising a lumen. See, e.g., FIG. 4, structures (c) to (f) and (i) to (j).

16. The tubing of aspect 14, comprising three nonporous liners each comprising a lumen.

17. The tubing of aspect 14, comprising four or more nonporous liners each comprising a lumen.

18. The tubing of any of aspects 15-17, wherein each nonporous liner is separate from (not fused to) any other nonporous liner. See, e.g., FIG. 3.

19. The tubing of any of aspects 15-17, wherein each of the nonporous liners are fused or bonded to another nonporous liner, optionally with a layer of porous fluoropolymer between the liners to which the liners are fused or bonded. See, e.g., FIG. 2.

20. The tubing of aspects 15 or 18, wherein each of the nonporous liners are substantially in the form of a half-circle with a layer of porous fluoropolymer between the flat faces forming a “DD” structure that is optionally fused or bonded.

21. The tubing of aspects 19 or 20, wherein the layer of fluoropolymer between the liners has a density in the range of about 0.3 to about 1.9 grams per cubic centimeter (g/cc) (e.g., from about 0.3 to about 0.9 g/cc, from about 0.9 to about 1.4 g/cc, from about 1.4 to about 1.9 g/cc), which is selected independently of the fluoropolymer of the outer walls.

22. The tubing of any of aspects 19-21, wherein, when the nonporous liners are bonded, they are bonded by an adherent material (e.g., adhesive material or adherent polymer).

23. The tubing of aspect 22, wherein the adherent polymer comprises a thermoplastic polymer or thermoplastic fluoropolymer.

24. Tubing comprising: (i) an outer wall having an outer surface located on the external face of the tubing, (ii) a nonporous liner comprising two or more lumens, and (iii) a fluorinated liquid; wherein the outer wall is comprised of a porous fluoropolymer having pores, and at least a portion of the fluorinated liquid is absorbed into the pores, and a portion of the fluorinated liquid is located (adsorbed) on the outer surface of the tubing (forming a slippery surface). See, e.g., FIG. 4, structures (h) and (k) to (n).

25. The tubing of aspect 24, wherein the nonporous liner comprises three or more, or four or more, lumens, and optionally comprises one or more (e.g., two or more) infolds in the liner (e.g., that will form a lumen when coated with porous fluoropolymer (see, e.g., FIG. 4, structure (h), without an infold and FIG. 4, structures (k) to (n), with one or two infolds 28.)

26. The tubing of aspects 24-25, wherein the nonporous liner is formed from a single piece of polymeric material (e.g., a single piece of extruded polymeric material having two or more lumens, or three or more lumens).

27. The tubing of any preceding aspect, wherein the porous fluoropolymer is comprised of an expanded fluoropolymer or electrospun fluoropolymer.

28. The tubing of any preceding aspect, wherein the porous fluoropolymer is comprised of expanded polytetrafluoroethylene (ePTFE) or electrospun PTFE.

29. The tubing of any preceding aspect, wherein the porous fluoropolymer is comprised of polyvinylidene difluoride (e.g., expanded PVDF (“ePVDF”)).

30. The tubing of any preceding aspect, wherein the porous fluoropolymer has a density from about 0.3 to about 1.9 grams per cubic centimeter (g/cc).

31. The tubing of any preceding aspect, wherein the porous fluoropolymer has a density of about 0.3 to 0.4 g/cc or about 0.4 to about 0.5 g/cc.

32. The tubing of any of aspects 1 to 30, wherein the porous fluoropolymer has a density of about 0.5- to about 0.6 g/cc or about 0.6 to about 0.7 g/cc.

33. The tubing of any of aspects 1 to 30, wherein the porous fluoropolymer has a density of about 0.7 to about 0.8 g/cc or about 0.8 to about 0.9 g/cc.

34. The tubing of any of aspects 1 to 30, wherein the porous fluoropolymer has a density of about 0.9 to about 1.0 g/cc or about 1.0 to about 1.1 g/cc.

35. The tubing of any of aspects 1 to 30, wherein the porous fluoropolymer has a density of about 1.1 to about 1.2 g/cc or about 1.2 to about 1.3 g/cc.

36. The tubing of any of aspects 1 to 30, wherein the porous fluoropolymer has a density of about 1.3 to about 1.4 g/cc or about 1.4 to about 1.8 g/cc.

37. The tubing of any of aspects 1 to 29, comprising a porous fluoropolymer with a reservoir volume of about 0.1 cc/g to about 0.2 cc/g or about 0.2 cc/g to about 0.3 cc/g.

38. The tubing of any of aspects 1-29, comprising a porous fluoropolymer with a reservoir volume from about 0.2 cc/g to about 0.4 cc/g or about 0.3 cc/g to about 0.5 cc/g.

39. The tubing of any of aspects 1 to 29, comprising a porous fluoropolymer with a reservoir volume from about 0.4 cc/g to about 0.5 cc/g or about 0.5 cc/g to about 0.6 cc/g.

40. The tubing of any preceding aspect, wherein, when the tubing comprises one or more septa comprised of a porous fluoropolymer, the outer wall and the septa are comprised of independently selected porous fluoropolymers.

41. The tubing of aspect 40, wherein the independently selected fluoropolymers of the outer wall and at least one of the one or more septa differ in density and/or chemical composition.

42. The tubing of any preceding aspect, wherein the fluorinated liquid is selected from the group consisting of: perfluoropropane, perfluorobutane, perfluoropentane, perfluorohexane, perfluorooctane, perfluorodecalin, perfluoroperhydrophenanthrene, perfluorooctylbromide, perfluorotributylamine, perfluorotripentylamine, poly(hexafluoropropylene oxide), 1H,4H-perfluorobutane, 1H-perfluoropentane, HFA 134a™, HFA227ea™, methyl perfluorobutylether, methyl perfluoropropyl ether (3M Novec 7000™), 2,2,2-trifluoroethanol, perfluoro-poly-propylene oxide (Krytox oil), and combinations thereof; or, alternatively, selected from the group consisting of perfluoropropane, perfluorobutane, perfluoropentane, perfluorohexane, perfluorooctane, perfluorodecalin, perfluoroperhydrophenanthrene, perfluorooctylbromide, perfluorotributylamine, perfluorotripentyl amine, poly(hexafluoropropylene oxide) and combinations thereof.

43. The tubing of any of aspects 1 to 41, wherein the fluorinated liquid is a fluorocarbon, perfluorocarbon, or mixture of any thereof (e.g., a mixture of perfluorocarbons).

44. The tubing of any preceding aspect, wherein the fluorinated liquid comprises perfluorodecalin or perfluorotributylamine.

45. The tubing of any preceding aspect, wherein, when the tubing comprises one or more lumens, or two or more lumens, lined with a nonporous liner, each liner is comprised of a polymer selected independently for each liner from the group consisting of: polyurethanes, silicones, fluoropolymers, perfluoropolymers, and fluoropolymer and perfluoropolymer blends.

46. The tubing of aspect 45, wherein at least one liner is comprised of a polyurethane or a silicone.

47. The tubing of aspect 45, wherein at least one liner is comprised of PTFE, FEP, PFA, PVDF, EFEP, or ETFE.

48. The tubing of aspect 45, wherein at least one liner is comprised of PTFE or FEP.

49. The tubing of aspect 45, wherein at least one liner is comprised of PTFE.

50. The tubing of any preceding aspect, wherein, when the tubing comprises at least one nonporous liner, each nonporous liner has an independently selected wall thickness (e.g., at its thinnest point) from about 10 microns to about 160 microns.

51. The tubing of aspect 50, wherein at least one nonporous liner has a thickness in a range selected from about 10 microns to about 20 microns or about 20 microns to about 40 microns.

52. The tubing of aspect 50, wherein at least one nonporous liner has a thickness in a range selected from about 40 microns to about 60 microns or about 60 microns to about 80 microns.

53. The tubing of aspect 50, wherein at least one nonporous liner has a thickness in a range selected from about 80 microns to about 100 microns or about 100 microns to about 120 microns.

54. The tubing of any preceding aspect, comprising a porous fluoropolymer reservoir inside of the outer wall, wherein the pores of the porous fluoropolymer reservoir are in fluid communication with the pores of the porous fluoropolymer outer wall, but the porous fluoropolymer reservoir directly contacts only part (portion) of the outer wall. See, e.g., FIG. 4, structures (a) to (g) and (i) to (j), items 22 and 25.

55. The tubing of any preceding aspect, further comprising a reservoir of fluorinated liquid in addition to the fluorinated liquid absorbed into the pores of the outer wall or present on the outer surface of the tubing.

56. The tubing of aspect 55, wherein the reservoir of fluorinated liquid is located within a reservoir space inside of the tubing. See, e.g., FIG. 4, reservoir lumen (g), item 26, and structures (i) to (n) item 29.

57. The tubing of aspect 56, wherein the reservoir space is located within the outer wall and/or one or more septa of the tubing.

58. The tubing of aspect 56 or 57, wherein the reservoir space contacts a porous fluoropolymer of the tubing (e.g., the porous fluoropolymer of the outer wall), such that fluorinated liquid in the reservoir space is in fluid communication with the pores of the porous fluoropolymer.

59. The tubing of any of aspects 56 to 58, comprising a first (distal) end and a second (proximal) end at opposite ends of the tubing, wherein the reservoir space inside of the tubing is a lumen of the tubing (i.e., a lumen of the tubing extending all or part of the length of the tubing from the first to the second end), which is optionally lined or unlined, and forming (may be utilized as) a reservoir lumen.

60. The tubing of aspect 59, wherein the reservoir lumen is closed at or proximate to the first (distal) end (e.g., the reservoir lumen terminates at the first end or within the tubing proximate to the first end).

61. The tubing of aspect 59, wherein the reservoir lumen has a diameter that narrows at the first end.

62. The tubing of aspect 61, wherein the diameter of the reservoir narrows to a diameter (e.g., a pore) less than 10% or less than 5% of the lumen diameter.

63. The tubing of aspect 59, wherein the reservoir lumen comprises a valve at or proximate to the first (distal) end.

64. The tubing of aspect 63, wherein the valve at or proximate to the first (distal) end is a slit providing fluid communication between the reservoir lumen and the exterior of the tubing.

65. The tubing of aspect 64, wherein the slit remains substantially or completely closed when fluid pressure in the reservoir lumen equals fluid pressure exterior to the reservoir lumen at the location of the slit.

66. The tubing of any of aspects 59 to 65, wherein the reservoir lumen is open at the second (proximal) end of the reservoir lumen and is joined to and in fluid communication with the lumen of another tube.

67. The tubing of any of aspects 59 to 65, wherein the reservoir lumen is closed at or proximate to the second (proximal) end (e.g., the reservoir lumen terminates at the second end or within the tubing proximate to the second end).

68. The tubing of any of aspects 59 to 65, wherein the reservoir lumen is closed at the second (proximal) end by a proximal end stopper or septum, or by a valve through which fluorinated liquid may be added or removed from the reservoir lumen.

69. The tubing of aspect 68, wherein the proximal end stopper or septum is a self-sealing stopper or septum (e.g., through which a non-coring needle may be used to add or remove fluorinated liquid from the reservoir lumen).

70. The tubing of any preceding aspect further comprising one or more wires and/or rods (e.g., within the outer wall or within a septa between two or more lumens).

71. The tubing of any preceding aspect further comprising a radiopaque material and/or contrast agent permitting the tubing to be imaged using an X-ray-based technology.

72. The tubing of aspect 71, wherein at least a portion of the radiopaque material and/or contrast agent is printed on the catheter.

73. The tubing of aspect 71 or 72, wherein at least a portion of the radiopaque material and/or contrast agent is incorporated into the tubing (e.g., into a porous fluoropolymer or polymer used to form a liner of the tubing).

74. The tubing of any of aspects 71 to 73, wherein at least a portion of the radiopaque material and/or contrast agent is a metallic wire, or a radiopaque polymer.

75. The tubing of any of aspects 71 to 73, wherein at least a portion of the radiopaque material and/or contrast agent is arranged as one or more discrete drops or deposits in all or part of the tubing (e.g., in all or part of the outer wall or all or part of a septum between lumens).

76. The tubing of any of aspects 71 to 73, wherein at least a portion of the radiopaque material and/or contrast agent is an X-ray contrast agent within one or more lumens of the tubing.

77. The tubing of any preceding aspect, wherein the outer surface of the tubing is water repellant and has a water roll off angle less than 10° or less than 5°.

78. The tubing of any preceding aspect, wherein the outer surface of the tubing has a water contact angle at 22° C. greater than 70° or greater than 90° degrees.

79. A medical device comprising a section of tubing according to any of aspects 1 to 78.

80. The medical device according to aspect 79, wherein the medical device is either fully implantable (for implantation, e.g., as in the case of a shunt or a totally implantable venous access port) or partially implantable or partially insertable in a patient (e.g., has a portion that is not to be inserted into a patient such as in the case of most catheters).

81. The medical device according to aspect 79 or 80, wherein the medical device is partially implantable or insertable in a patient (has a portion that is for implantation or insertion in the patient), and the portion that is partially implantable or insertable in the patient is comprised of a section of tubing of the present disclosure.

82. The medical device of aspect 79 or 80, wherein the medical device is selected from the group consisting of catheters, cannulas, drains, shunts, totally implantable venous access ports, and ports.

83. The medical device of any of aspects 79 to 82, wherein the medical device is a catheter (e.g., a HD catheter).

84. The catheter of any of aspects 82 to 83, wherein the catheter comprises (i) a distal end portion terminating in a tip forming the distalmost end of the catheter for insertion into a patient, and (ii) a proximal end portion; wherein:
the distal end portion comprises a section of tubing having one two, three, or more lumens according to any of aspects 1 to 78, with at least one lumen optionally comprising a nonporous polymer liner, provided that when the catheter has a single lumen it is lined by a nonporous liner;
the proximal end portion comprises one or more lumens, at least one of which is in fluid communication with at least one of the one, two, three, or more lumens of the distal end portion, and the proximal end portion comprises one or more fittings that form fluid tight seals with the one or more lumens of the proximal end portion; and
the tip has one or more openings located either in the outer wall and/or at the tip providing fluid communication between one, two, three, or more lumens of the distal end portion and the exterior of the catheter.

85. The medical device of any of aspects 79 to 80, wherein the device is a shunt.

86. The device of aspect 85, wherein the shunt comprises: (i) a distal end portion terminating in a tip forming the distalmost end of the shunt, and (ii) a proximal end portion, wherein:
the distal end portion comprises a section of tubing having one two, three, or more lumens according to any of aspects 1 to 78, with at least one lumen optionally comprising a nonporous polymer liner, provided that when the catheter has a single lumen it is lined by a nonporous liner;
the proximal end portion comprises one or more lumens, at least one of which is in fluid communication with at least one of the one, two, three, or more lumens of the distal end portion; and the tip has one or more openings located either in the outer wall and/or at the tip providing fluid communication between one, two, three, or more lumens of the distal end portion and the exterior of the catheter.

87. The shunt of aspect 86, wherein the shunt comprises a shunt valve in fluid communication with at least one lumen in the distal end portion and at least one lumen in the proximal end portion.

88. The catheter or the shunt of any of aspects 83 to 87, wherein each lumen in the distal end portion is in fluid communication with a lumen in the proximal end portion.

89. The catheter or the shunt of aspect 88, wherein the catheter or shunt has a single lumen.

90. The catheter or the shunt of aspect 88, wherein the catheter or shunt has two lumens.

91. The catheter or the shunt of aspect 88, wherein the catheter or shunt has three lumens.

92. The catheter or the shunt of any of aspects 82 to 91, wherein the section of tubing according to any of aspects 1-78 is an expanded fluoropolymer or electrospun fluoropolymer.

93. The catheter or the shunt of any of aspects 82 to 91, wherein the section of tubing according to any of aspects 1-78 comprises polytetrafluoroethylene (ePTFE).

94. The catheter or the shunt of any of aspects 82 to 91, wherein the section of tubing according to any of aspects 1-78 comprises polyvinylidene difluoride (e.g., ePVDF).

95. The catheter or the shunt of any of aspects 82 to 94, wherein the porous fluoropolymer has a density from 0.3 to 1.9 g/cc.

96. The catheter or the shunt of any of aspects 82 to 94, wherein the porous fluoropolymer has a density of 0.3 to 0.4 g/cc or 0.4 to 0.5 g/cc.

97. The catheter or the shunt of any of aspects 82 to 94, wherein the porous fluoropolymer has a density of 0.5 to 0.6 g/cc or 0.6 to 0.7 g/cc.

98. The catheter or the shunt of any of aspects 82 to 94, wherein the porous fluoropolymer has a density of 0.7 to 0.8 g/cc or 0.8 to 0.9 g/cc.

99. The catheter or the shunt of any of aspects 82 to 94, wherein the porous fluoropolymer has a density of 0.9 to 1.0 g/cc or 1.0 to 1.1 g/cc.

100. The catheter or the shunt of any of aspects 82 to 94, wherein the porous fluoropolymer has a density of 1.1 to 1.2 g/cc or 1.2 to 1.3 g/cc.

101. The catheter or the shunt of any of aspects 82 to 94, wherein the porous fluoropolymer has a density of 1.3 to 1.4 g/cc or 1.4 to 1.8 g/cc.

102. The catheter or the shunt of any of aspects 82 to 101, wherein the fluorinated liquid comprises one or more liquids selected from the group consisting of: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-perfluoropentane, HFA 134a™, HFA227ea™, methyl perfluorobutylether, methyl perfluoropropyl ether (3M Novec 7000™), 2,2,2-trifluoroethanol, perfluoro-poly-propylene oxide (Krytox oil), and combinations thereof.

103. The catheter or the shunt of any of aspects 82 to 101, wherein the fluorinated liquid comprises one or more liquids selected from the group consisting of: perfluoropropane, perfluorobutane, perfluoropentane, perfluorohexane, perfluorooctane, perfluorodecalin, perfluoroperhydrophenanthrene, perfluorooctylbromide, perfluorotributylamine, perfluorotripentyl amine, poly(hexafluoropropylene oxide) and combinations thereof.

104. The catheter or the shunt of any of aspects 82 to 101, wherein the fluorinated liquid comprises a fluorocarbon, perfluorocarbon, or mixture of any thereof (e.g., a mixture of perfluorocarbons).

105. The catheter or the shunt of any of aspects 82 to 101, wherein the fluorinated liquid comprises perfluorodecalin or perfluorotributylamine.

106. The catheter or the shunt of any of aspects 82 to 101, wherein the fluorinated liquid comprises perfluorodecalin.

107. The catheter or the shunt of any of aspects 82 to 106, wherein, when the distal end portion comprises a section of tubing according to any of aspects 1 to 78 having two lumens (a dual lumen catheter), one or both of the lumens comprises a nonporous polymer liner.

108. The catheter or the shunt of any of aspects 82 to 107, wherein when the catheter has two lumens (i.e., is a dual lumen catheter) each lumen has a cross section (normal to the axis of the tubing) that is a half-circle or substantially a half-circle arranged with the flat faces of the half-circles oriented toward each other to form a “DD” structure.

109. The catheter or the shunt of any of aspects 82 to 106, wherein, when the distal end portion comprises a section of tubing according to any of aspects 1 to 78 having three or more lumens, one or more, or two or more of the lumens comprise a nonporous polymer liner.

110. The catheter or the shunt of any of aspects 82 to 109, wherein each lumen comprises a liner of a nonporous polymer.

111. The catheter or the shunt of any of aspects 107 to 110, wherein each liner is comprised of a polymer selected independently from the group consisting of: polyurethanes, silicones, fluoropolymers, perfluoropolymers, and fluoropolymer and perfluoropolymer blends.

112. The catheter or the shunt of any of aspects 107 to 110, wherein each liner is comprised of a polymer selected independently from the group consisting of: PTFE, FEP, PFA, PVDF, EFEP, or ETFE.

113. The catheter or the shunt of any of aspects 107 to 110, wherein each liner is comprised of a polymer selected independently from the group consisting of: PTFE or FEP.

114. The catheter or the shunt of any of aspects 107 to 110, wherein each liner is of PTFE or a polyurethane.

115. The catheter of any preceding aspect, wherein each liner has a thickness selected independently within the range of 10 microns to 160 microns.

116. A catheter or a shunt comprising a section of tubing, wherein the section of tubing is comprised of: (i) at least two lumens separated by septa (walls between the lumens), (ii) an outer wall having an outer surface located on the external face of the tubing, and (iii) a fluorinated liquid; wherein the outer wall and the septa are comprised of a porous fluoropolymer having pores, and at least a portion of the fluorinated liquid is absorbed into the pores (of both the septa and the outer wall), and a portion of the fluorinated liquid is located (adsorbed) on the outer surface of the tubing (forming a slippery surface). See, e.g., aspects 1-13 and their dependent aspects.

117. A catheter or a shunt comprising a section of tubing, wherein the section of tubing is comprised of: (i) an outer wall having an outer surface located on the external face of the tubing, (ii) at least one nonporous liner within and contacting the outer wall, the liner comprising a lumen, and (iii) a fluorinated liquid; wherein the outer wall is comprised of a porous fluoropolymer having pores, and at least a portion of the fluorinated liquid is absorbed into the pores, and a portion of the fluorinated liquid is located (adsorbed) on the outer surface of the tubing (forming a slippery surface). See, e.g., aspects 14-23 and their dependent aspects.

118. A catheter or a shunt comprising a section of tubing, wherein the section of tubing is comprised of: (i) an outer wall having an outer surface located on the external face of the tubing, (ii) a nonporous liner comprising two or more lumens, and (iii) a fluorinated liquid; wherein the outer wall is comprised of a porous fluoropolymer having pores, and at least a portion of the fluorinated liquid is absorbed into the pores, and a portion of the fluorinated liquid is located (adsorbed) on the outer surface of the tubing (forming a slippery surface). See e.g., aspects 24-26 and their dependent aspects.

119. The catheter or shunt of any of aspects 116 to 118, wherein the porous fluoropolymer is porous PTFE or porous PVDF (e.g., ePVDF).

120. The catheter or shunt of aspect 119 wherein the porous PTFE is expanded PTFE (ePTFE) or electrospun PTFE.

121. The catheter or shunt of any of aspects 116 to 120, further comprising a reservoir lumen that is optionally filled with fluorinated liquid.

122. The catheter or shunt of any of aspects 116 to 121, wherein at least one lumen other than the reservoir lumen is lined with a nonporous liner.

123. The catheter or shunt of any of aspects 116 to 122, wherein the fluorinated liquid comprises perfluorodecalin or perfluorotributylamine.

124. A method comprising partially or completely implanting or inserting a medical device of any of aspects 79 to 123 in a mammalian patient (e.g. a catheter, shunt, cannula, drain, or port, such as a totally implantable venous access port).

125. A method comprising partially or completely implanting or inserting a shunt according to any of aspects 85 to 123 in a mammalian patient.

126. The method of aspect 126, wherein the shunt is a hydrocephalus shunt.

127. A method comprising partially or completely implanting or inserting a catheter according to any of aspects 83, 84 or 88 to 123 in a mammalian patient.

128. The method of aspect 127, wherein the catheter (the distal end of a catheter) is inserted into an artery or vein of a mammalian patient.

129. The method of any of aspects 127 or 128, wherein the patient is suffering from a renal or cardiovascular disease or disorder.

130. A method for treating a renal or cardiovascular disease or disorder in a patient in need thereof, the method comprising implanting a catheter of any of aspects 83, 84 or 88 to 123 into a vein or artery of the patient to access blood flow or a tissue.

131. The method of any of aspects 129 or 130, wherein the renal disease or disorder is a partial or complete kidney failure.

132. The method of any of aspects 129 to 131, wherein the catheter is a hemodialysis catheter.

133. The method of any of claim 129 or 130, wherein the cardiovascular disease or disorder is selected from the group consisting of: restenosis, coronary artery disease, atherosclerosis, atherogenesis, cerebrovascular disease, angina, ischemic disease, congestive heart failure, pulmonary edema associated with acute myocardial infarction, thrombosis, platelet aggregation, platelet adhesion, smooth muscle cell proliferation, a vascular or non-vascular complication associated with the use of a medical device, a wound associated with the use of a medical device, vascular or non-vascular wall damage, peripheral vascular disease, and neointimal hyperplasia following percutaneous transluminal coronary angiograph.

134. The method of any of aspects 124 to 133, further comprising filling at least one lumen of the medical device with a solution comprising a fluorinated liquid either before or at the time of inserting or implanting the medical device (e.g., catheter or shunt) in a patient.

135. The method of any of aspects 124 to 134, further comprising filling at least one lumen of the medical device (e.g., shunt or catheter) with a solution comprising a fluorinated liquid after inserting or implanting the medical device (e.g., catheter or shunt) in a patient.

136. The method of any of aspects 124 to 135, further comprising filling with a locking solution (e.g., locking off) at least one lumen of the medical device during all or part of the time when the medical device is not used to administer a fluid to the patient and/or withdraw a fluid from the patient.

137. The method of any of aspects 124 to 136, wherein, when the medical device comprises a reservoir lumen, the fluorinated liquid in the reservoir is replenished after the medical device is inserted or implanted in the patient.

138. The method of aspect 137, wherein the reservoir lumen is sealed at the distal end inserted or implanted in the patient.

139. The method of aspect 137, wherein the reservoir lumen has an opening or valve allowing some or all of the fluorinated liquid in the reservoir lumen to be released into the patient.

140. The method of any of aspects 124 to 139, wherein the patient (or subject) is human.

141. A method of making tubing comprising a porous fluoropolymer outer wall and nonporous fluoropolymer lined lumens comprising the steps of:
(i) bringing two or more nonporous polymer tubes having lumens into contact, optionally with an intervening layer of material,
(ii) bonding (e.g., by sintering or adhering) the two or more nonporous polymer tubes (along with any intervening material present) together to form an inner liner assembly, and
(iii) covering the inner liner assembly with a porous fluoropolymer outer wall (e.g., either by extrusion forming of a layer of porous fluoropolymer over the inner liner assembly, or by inserting the inner liner assembly into a tube of porous fluoropolymer, optionally followed by sintering the porous fluoropolymer and inner liner assembly together).

142. The method of aspect 141, wherein the two or more (e.g., three or more) nonporous polymer tubes are formed into a shape (e.g., by passage over a mandrel or heated mandrel) substantially conforming to the shape they will have in the inner liner assembly prior to bringing the tubes into contact or into contact with each other and/or with any intervening material that may be present.

143. The method of aspect 142, wherein the tubing comprises two lumens and the nonporous polymer tubes are shaped so that they have a D-shape (half-circle) comprising a flat face when sectioned perpendicular to the tube's longitudinal axis (the tube's longitudinal axis running parallel to the tube's lumens).

144. The method of aspect 143, wherein the flat faces are brought into contact to form the inner liner assembly, optionally with the intervening layer of material interposed between the flat faces.

145. The method of any of aspects 141 to 144, wherein the intervening layer of material is interposed between the nonporous tubes.

146. The method of aspect 145, wherein the intervening layer of material comprises a porous fluoropolymer.

147. A method of forming tubing comprising an outer wall and two or more lumens separated by septa, the method comprising extrusion forming the outer wall and at least one septum, wherein the outer wall and the at least one septum (each septum) separating the lumens is comprised of a porous fluoropolymer.

148. The method of aspect 147, wherein the tubing comprises three or more (e.g., four or more) lumens separated by septa.

149. The method of aspect 147 or 148, further comprising: (i) forming a section of a nonporous polymeric tube into a liner for a corresponding lumen of the tubing by inserting a mandrel within the nonporous tube resulting in a liner substantially conforming to the shape of the corresponding lumen of the tubing; (ii) inserting the tubing and mandrel into the corresponding lumen; and (iii) withdrawing the mandrel.

150. The method of aspect 149, wherein (i) two or more sections of independently selected nonporous polymeric tube are formed into liners for corresponding lumens of the tubing on separate mandrels; (ii) the formed liners and mandrels are inserted into the corresponding lumens of the tubing either simultaneously or sequentially; and (iii) the mandrels are withdrawn either simultaneously or sequentially.

151. The method of any of aspects 141 to 150, wherein the porous fluoropolymer is ePTFE or ePVDF.

152. The method of any of aspects 141 to 150, further comprising applying a liquid comprising, consisting essentially of, or consisting of one or more fluorinated liquids to the tubing.

153. The method of aspect 152, wherein the fluorinated liquid is perfluorodecalin or perfluorotributylamine.

### 6.0 EXAMPLES

### Example 1

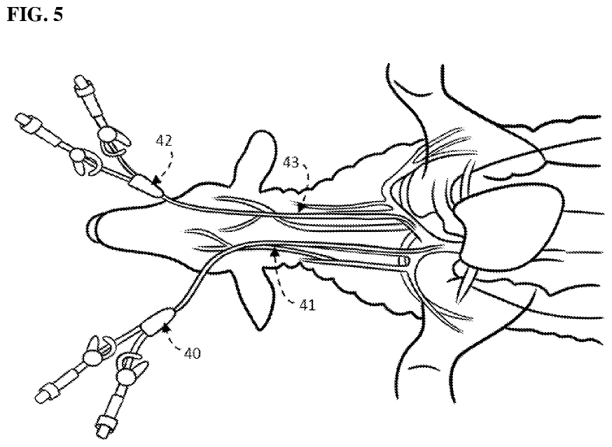

A test catheter having a section of unlined dual lumen ePTFE tubing (ePTFE density 1.2 g/cc) with DD structure at its distal (implanted) end was prepared by an extrusion process. The dimensions of the ePTFE section of the catheter were 5 mm diameter, wall thickness approximately 0.64 mm, and septum thickness approximately 0.9 mm. The finished catheter was produced by attaching a bifurcation tube, clamps 8 and hubs. The test catheter 40 was treated with perfluorodecalin and implanted into the right jugular vein 41 of a domestic sheep. A Medtronic Palindrome® catheter control 42 was implanted into the left jugular vein 43 of the same domestic sheep for direct comparison (FIG. 5). Both catheters were locked with sterile heparinized saline (100 U/mL) locking solution before implantation. The test catheter was flushed with 1.9 mL perfluorodecalin and the control catheter was flushed with saline after implantation. Both catheters were inserted under fluoroscopic guidance and were found to extend distally with both catheter tips located in the superior vena cava (SVC) without touching each other. The proximal ends of both catheters with Luer-lock fittings (“hubs”) were coiled inside a subcutaneous pocket adjacent to the point of insertion (to prevent the sheep from dislodging or otherwise disturbing the catheters) and were sutured to a muscle layer using non-absorbable polypropylene sutures.

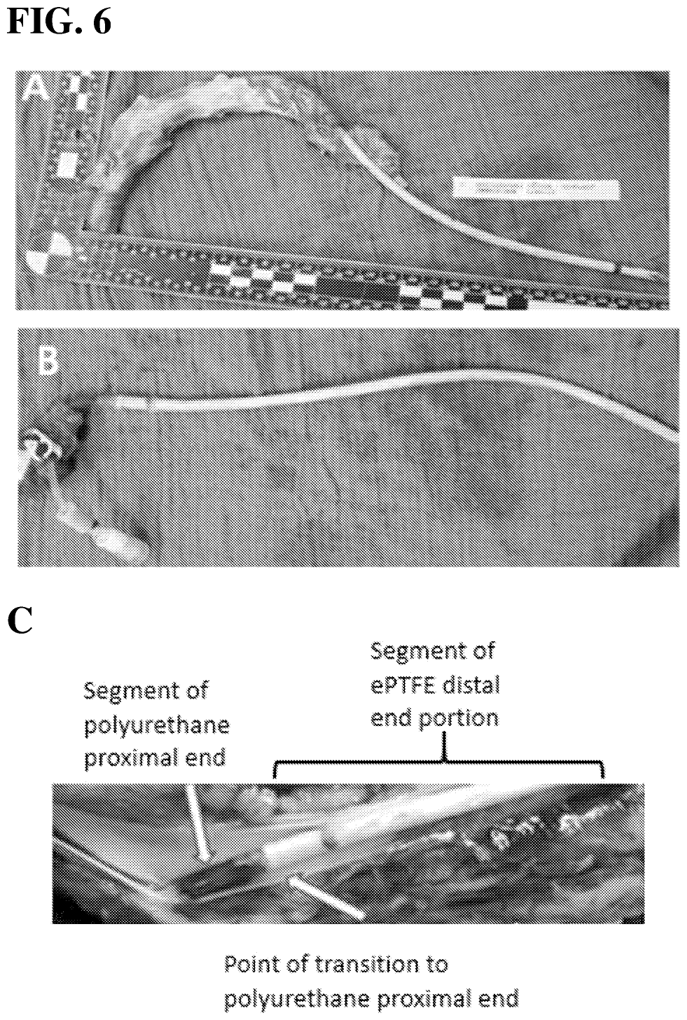

After 90 days of implantation, the sheep was euthanized following an IACUC-approved protocol. Both the test catheter and Palindrome® catheter were isolated with surround tissues including the SVC and implanted vessels in one continuous piece. The surrounding tissue was dissected to expose the fibrous sheath and any thrombi that formed. Coverage of the device by fibrin sheathing and/or thrombi formation was calculated by measuring each section of sheath and/or thrombus with a calibrated caliper. The total coverage of an identified area was calculated by taking the length of each section of fibrin sheath and/or thrombus that formed (as indicated) and using the circumference of the catheter to calculate the area covered. Where a section of fibrin sheath or thrombus did not completely wrap around the device, the portion covered was calculated by its length along the tube and fraction of the circumference covered. The percentage was then calculated by dividing the area covered by the total area of the device implanted and multiplying by 100. While Medtronic Palindrome® control showed 62% fibrin sheath formation, TLP HDC showed only 5.7% fibrin sheath (see FIG. 6). Catheter tips were inspected after opening both veins. The Palindrome® control tip (see FIG. 7 at A) were completely occluded with thrombus and tissue ingrowth, but the tip of the test catheter (see FIG. 7 at B) was completely open. Catheter hubs from the test HDC (prepared from solid PTFE and treated with perfluorodecalin) were sonicated in PBS to dislodge any bacterial biofilm if formed. Subsequent microbiological evaluation reported zero CFU after 72 h of incubation of three serial dilutions (10-1, 10-2, and 10-3). Biofilm evaluation of the control Palindrome® catheter hubs was precluded by the heavy fibrin sheath (see FIG. 6 at A). The surface of the PTFE and perfluorodecalin treated HDC was visually hydrophobic, with water beading on its surface, upon removal of the catheter from the sheep.

### Example 2

A section of tubing used to form the catheter in Example 1 was wrapped around a series of rods of increasingly smaller diameter (perpendicular to the longitudinal axis of the rods). The tubing could be wrapped around rods as small as 0.7 cm without causing either lumen to collapse (i.e., the section of tubing “kinking”) so that the flow of liquid was not blocked.

1. A catheter, cannula, drain, shunt, port, or totally implantable venous access port comprising a section of tubing, wherein the section of tubing comprises: (i) at least two lumens separated by septa separating the lumens, (ii) an outer wall having an outer surface located on the external face of the tubing, and (iii) a fluorinated liquid,
wherein
(a) the outer wall and the septa are comprised of a porous fluoropolymer having pores, and
(b) at least a portion of the fluorinated liquid is absorbed into the pores of both the septa and the outer wall, and a portion of the fluorinated liquid is located on the outer surface of the tubing, and
(c) the fluorinated liquid in the pores of the septa, pores of the outer wall, and on the outer surface are in fluid communication and can flow between the pores and the outer surface.

2. A catheter, cannula, drain, shunt, port, or totally implantable venous access port comprising a section of tubing, wherein the section of tubing comprises: (i) an outer wall having an outer surface located on the external face of the tubing, (ii) at least one nonporous liner within and contacting the outer wall, the liner comprising a lumen, and (iii) a fluorinated liquid; wherein
(a) the outer wall is comprised of a porous fluoropolymer having pores,
(b) at least a portion of the fluorinated liquid is absorbed into the pores, and a portion of the fluorinated liquid is located on the outer surface of the tubing, and
(c) the fluorinated liquid in the pores of the outer wall and on the outer surface are in fluid communication and can flow between the pores and the outer surface.

3. A catheter, cannula, drain, shunt, port, or totally implantable venous access port comprising a section of tubing, wherein the section of tubing comprises: (i) an outer wall having an outer surface located on the external face of the tubing, (ii) a nonporous liner comprising two or more lumens, and (iii) a fluorinated liquid; wherein
(a) the outer wall is comprised of a porous fluoropolymer having pores,
(b) at least a portion of the fluorinated liquid is absorbed into the pores, and a portion of the fluorinated liquid is located (adsorbed) on the outer surface of the tubing, and
(c) the fluorinated liquid in the pores of the outer wall and on the outer surface are in fluid communication and can flow between the pores and the outer surface.

4. The catheter, cannula, drain, shunt, port, or totally implantable venous access port of any of claims 1 to 3, wherein the porous fluoropolymer comprises a porous perfluoropolymer.

5. The catheter, cannula, drain, shunt, port, or totally implantable venous access port of any of claims 1 to 3, wherein the porous fluoropolymer is porous PTFE or porous PVDF.

6. The catheter, cannula, drain, shunt, port, or totally implantable venous access port of claim 5, wherein the porous PTFE is expanded PTFE (ePTFE) or electrospun PTFE.

7. The catheter, cannula, drain, shunt, port, or totally implantable venous access port of claim 6, wherein the fluorinated liquid comprises perfluorodecalin or perfluorotributylamine.

8. The catheter, cannula, drain, shunt, port, or totally implantable venous access port of claim 7, wherein at least one lumen is lined with a nonporous liner.

9. The catheter, cannula, drain, shunt, port, or totally implantable venous access port of claim 7, further comprising a reservoir lumen that is optionally filled with fluorinated liquid.

10. The catheter or cannula of claim 7.

11. The drain, shunt, port, or totally implantable venous access port of claim 7.

12. A method comprising partially or completely implanting or inserting a catheter, cannula, drain, shunt, port, or totally implantable venous access port of claim 5 in a mammalian patient.

13. A method comprising partially or completely implanting or inserting a catheter, cannula, drain, shunt, port, or totally implantable venous access port of claim 7 in a mammalian patient.

14. A method comprising partially or completely implanting or inserting a catheter, cannula, drain, shunt, port, or totally implantable venous access port of claim 8 in a mammalian patient.

15. A method comprising partially or completely implanting or inserting a shunt or catheter of claim 5 in a mammalian patient, wherein the shunt is a hydrocephalus shunt and the catheter is a hemodialysis catheter.

16. A method comprising partially or completely implanting or inserting a shunt or catheter of claim 7 in a mammalian patient, wherein the shunt is a hydrocephalus shunt and the catheter is a hemodialysis catheter.

17. A method comprising partially or completely implanting or inserting shunt or catheter of claim 8 in a mammalian patient, wherein the shunt is a hydrocephalus shunt and the catheter is a hemodialysis catheter.

18. The method of claim 16, wherein the hemodialysis catheter has a distal end, and the distal end of the catheter is inserted into an artery or vein of a mammalian patient.

19. The method of claim 13, wherein the patient is suffering from a renal or cardiovascular disease or disorder.

20. The method of claim 19, wherein the patient is suffering from a cardiovascular disease or disorder selected from the group consisting of: restenosis, coronary artery disease, atherosclerosis, atherogenesis, cerebrovascular disease, angina, ischemic disease, congestive heart failure, pulmonary edema associated with acute myocardial infarction, thrombosis, platelet aggregation, platelet adhesion, and smooth muscle cell proliferation.

21. A method for treating a renal or cardiovascular disease or disorder in a patient in need thereof, the method comprising implanting a catheter of claim 5, into a vein or artery of the patient.

22. The method of claim 21, wherein the renal disease or disorder is partial or complete kidney failure and the catheter is a hemodialysis catheter.

23. The method of claim 5, further comprising filling at least one lumen of the catheter, cannula, drain, shunt, port, or totally implantable venous access port with a solution comprising a fluorinated liquid before, at the time of inserting, or after insertion in the patient.

24. The method of claim 7, further comprising filling at least one lumen of the catheter, cannula, drain, shunt, port, or totally implantable venous access port with a solution comprising a fluorinated liquid before, at the time of inserting, or after insertion in the patient.

25. The method of claim 8, further comprising filling at least one lumen of the catheter, cannula, drain, shunt, port, or totally implantable venous access port with a solution comprising a fluorinated liquid before, at the time of inserting, or after insertion in the patient.

26. The method of claim 15, further comprising filling at least one lumen of the catheter or shunt with a solution comprising a fluorinated liquid before, at the time of inserting, or after insertion in the patient.

27. The method of claim 16, further comprising filling at least one lumen of the catheter or shunt with a solution comprising a fluorinated liquid before, at the time of inserting, or after insertion in the patient.

28. The method of claim 17, further comprising filling at least one lumen of the catheter or shunt with a solution comprising a fluorinated liquid before, at the time of inserting, or after insertion in the patient.

29. The method of claim 21, further comprising filling at least one lumen of the catheter with a solution comprising a fluorinated liquid before, at the time of inserting, or after insertion in the patient.

30. The method of claim 22, further comprising filling at least one lumen of the hemodialysis catheter with a solution comprising a fluorinated liquid before, at the time of inserting, or after insertion in the patient.

31. A method of making tubing comprising a porous fluoropolymer outer wall and nonporous fluoropolymer lined lumens, comprising the steps of:
(i) bringing two or more nonporous polymer tubes having lumens into contact, optionally with an intervening layer of material,
(ii) bonding the two or more nonporous polymer tubes and any intervening material present together to form an inner liner assembly, and
(iii) covering the inner liner assembly with a porous fluoropolymer outer wall.

32. The method of claim 31, wherein the two or more nonporous polymer tubes are formed into a shape substantially conforming to the shape they will have in the inner liner assembly prior to bringing the tubes into contact with each other and/or into contact with any intervening material that may be present.

33. Tubing comprising: (i) at least two lumens separated by septa, (ii) an outer wall having an outer surface located on the external face of the tubing, and (iii) a fluorinated liquid; wherein
(a) the outer wall and the septa are comprised of a porous fluoropolymer having pores,
(b) at least a portion of the fluorinated liquid is absorbed into the pores of both the septa and the outer wall, and a portion of the fluorinated liquid is located on the outer surface of the tubing, and
(c) the fluorinated liquid in the pores of the septa, pores of the outer wall, and on the outer surface are in fluid communication and can flow between the pores and the outer surface.

34. Tubing comprising: (i) an outer wall having an outer surface located on the external face of the tubing, (ii) at least one nonporous liner within and contacting the outer wall, the liner comprising a lumen, and (iii) a fluorinated liquid; wherein
(a) the outer wall is comprised of a porous fluoropolymer having pores,
(b) at least a portion of the fluorinated liquid is absorbed into the pores, and a portion of the fluorinated liquid is located on the outer surface of the tubing, and
(c) the fluorinated liquid in the pores of the outer wall and on the outer surface are in fluid communication and can flow between the pores and the outer surface.

35. Tubing comprising: (i) an outer wall having an outer surface located on the external face of the tubing, (ii) a nonporous liner comprising two or more lumens, and (iii) a fluorinated liquid; wherein
(a) the outer wall is comprised of a porous fluoropolymer having pores,
(b) at least a portion of the fluorinated liquid is absorbed into the pores, and a portion of the fluorinated liquid is located (adsorbed) on the outer surface of the tubing, and
(c) the fluorinated liquid in the pores of the outer wall and on the outer surface are in fluid communication and can flow between the pores and the outer surface.

36. A medical device comprising a section of tubing according to any of claims 33 to 35.

37. The medical device according to claim 36, wherein the medical device is either fully implantable, or partially implantable or partially insertable, in a patient.

38. The medical device according to claim 37, wherein the medical device is partially implantable or insertable in a patient, and the portion that is partially implantable or insertable in the patient is comprised of a section of tubing of the present disclosure.

39. The medical device of claim 37, wherein the medical device is selected from the group consisting of catheters, cannulas, drains, shunts, totally implantable venous access ports, and ports.

40. The medical device of claim 37, wherein the medical device is a catheter.

41. The catheter of claim 40, wherein the catheter comprises (i) a distal end portion terminating in a tip forming the distalmost end of the catheter for insertion into a patient, and (ii) a proximal end portion; wherein:
the distal end portion comprises a section of tubing having one two, three, or more lumens, with at least one lumen optionally comprising a nonporous polymer liner, provided that when the catheter has a single lumen it is lined by a nonporous liner;
the proximal end portion comprises one or more lumens, at least one of which is in fluid communication with at least one of the one, two, three, or more lumens of the distal end portion, and the proximal end portion comprises one or more fittings that form fluid tight seals with the one or more lumens of the proximal end portion; and
the tip has one or more openings located either in the outer wall and/or at the tip providing fluid communication between one, two, three, or more lumens of the distal end portion and the exterior of the catheter.

42. A method of forming tubing comprising an outer wall and two or more lumens separated by septa, the method comprising extrusion forming the outer wall and at least one septum, wherein the outer wall and the at least one septum separating the lumens is comprised of a porous fluoropolymer.

43. The method of claim 42, wherein the tubing comprises three or more (e.g., four or more) lumens separated by septa.

44. The method of claim 43, further comprising: (i) forming a section of a nonporous polymeric tube into a liner for a corresponding lumen of the tubing by inserting a mandrel within the nonporous tube resulting in a liner substantially conforming to the shape of the corresponding lumen of the tubing; (ii) inserting the tubing and mandrel into the corresponding lumen; and (iii) withdrawing the mandrel.

45. The method of claim 44, wherein (i) two or more sections of independently selected nonporous polymeric tube are formed into liners for corresponding lumens of the tubing on separate mandrels; (ii) the formed liners and mandrels are inserted into the corresponding lumens of the tubing either simultaneously or sequentially; and (iii) the mandrels are withdrawn either simultaneously or sequentially.
